(19) United States
(12) Patent Application Publication
(10) Pub. No.: US 2023/0176069 A1
(43) Pub. Date: Jun. 8, 2023

(54) MITIGATING CONCENTRATION EFFECTS OF ANTIBODY DIGESTION FOR COMPLETE SEQUENCE COVERAGE

(71) Applicant: University of Virginia Patent Foundation, Charlottesville, VA (US)

(72) Inventors: Donald F. Hunt, Charlottesville, VA (US); Jeffrey Shabanowitz, Charlottesville, VA (US); Robert Anthony D’Ippolito, Frederick, MD (US); Maria C. Panepinto, Charlottesville, VA (US); Keira Mahoney, Charlottesville, VA (US)

(21) Appl. No.: 17/796,203

(22) PCT Filed: Jan. 28, 2021
(86) PCT No.: PCT/US2021/015527

§ 371 Date: Jul. 28, 2022

Related U.S. Application Data
(60) Provisional application No. 62/966,731, filed on Jan. 28, 2020.

(51) Int. Cl. G01N 33/68; C07K 16/28; C07K 16/30

(52) U.S. Cl. CPC G01N 33/6857; C07K 16/30; C07K 16/2809; C07K 2317/31

(57) ABSTRACT

The presently disclosed subject matter relates to a method for characterizing a protein. The method can comprise disposing a protein in a digestion buffer; disposing a hydrolyzing agent inhibitor in the digestion buffer; passing the digestion buffer comprising said protein and said hydrolyzing agent inhibitor through a reaction chamber comprising at least one hydrolyzing agent, wherein said protein contacts said hydrolyzing agent in the presence of said inhibitor and is present in the chamber for a period of time (t) sufficient to produce protein fragments and provide digestion of said protein in the chamber, wherein the passing of the digestion buffer comprising the protein and the hydrolyzing agent inhibitor through the chamber is done at an adjustable flow rate; and performing multi-segment liquid chromatography tandem mass spectrometry (LC MS/MS) to characterize the protein.

### CROSS REFERENCE TO RELATED APPLICATIONS

This application claims benefit of U.S. Provisional Application Serial No. 62/966,731 filed Jan. 28, 2020, the disclosure of which is incorporated herein by reference in its entirety.

### GRANT STATEMENT

This invention was made with government support under Grant No. GM037537 awarded by the National Institutes of Health. The government has certain rights in the invention.

### REFERENCE TO SEQUENCE LISTING SUBMITTED ELECTRONICALLY

The content of the electronically submitted sequence listing in ASCII text file (Name: 3062-105_PCT.ST25.txt; Size: 26 kilobytes; and Date of Creation: Jan. 28, 2021) filed with the application is incorporated herein by reference in its entirety.

### TECHNICAL FIELD

The presently disclosed subject matter relates to methods of analyzing proteins, including bispecific antibodies. The methods can involve disposing a protein in a digestion buffer; disposing a hydrolyzing agent inhibitor in the digestion buffer; passing the digestion buffer comprising the protein and the hydrolyzing agent inhibitor through a reaction chamber comprising at least one hydrolyzing agent, wherein said protein contacts said hydrolyzing agent in the presence of said inhibitor and is present in the chamber for a period of time (t) sufficient to produce protein fragments and provide digestion of said protein in the chamber, wherein the passing of the digestion buffer comprising the protein and the hydrolyzing agent inhibitor through the chamber is done at an adjustable flow rate; and performing multi-segment liquid chromatography tandem mass spectrometry (LC MS/MS) to characterize the protein.

### BACKGROUND

The determination of a protein’s primary structure is useful in countless biological applications, particularly in drug development. For instance, accurate identification of the primary structure of antibodies is useful to their safety and effectiveness as therapeutic agents. The number of antibodies approved as biologic therapeutic drugs has been increasing in recent years.¹ Bispecific antibodies (bsAbs) are a class of antibody therapeutics being heavily researched.²⁻⁵ While monospecific antibodies are produced naturally in the body and target a single epitope with both variable regions, bsAbs are highly engineered molecules that target a different epitope with each variable region.⁶ A wide variety of bsAb structures have been engineered; some mimic the IgG structure of monoclonal antibodies while others comprise a single chain containing two variable regions connected by a linker.⁶ Extensive characterization of these bsAbs is helpful to ensure their correct production.

Mass spectrometry is the principle method for determining the primary structure of antibodies.^{7,8} Typically, proteins are digested with trypsin overnight then analyzed by LC-MS/MS for peptide identification. The peptides observed are then stitched back together following a database search to reconstruct the original protein sequence. However, trypsin digests often result in incomplete sequence coverage because the generated peptides do not overlap in sequence and are sometimes not observed due to their extreme high or low hydrophobicity.⁹ Additional specific protease digestions can be necessary to unambiguously confirm the protein sequence.^{10,11} Overall, this process of multiple specific digestions can take up to 18 hours for the digestion plus 4-8 additional hours for instrument analysis to confirm the sequence.

Accordingly, there is an ongoing need in the art to provide additional methods of characterizing proteins, such as bispecific antibodies, e.g., to determine their primary structure, particularly those methods that can be performed in less time.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

In some embodiments, the presently disclosed subject matter provides a method for characterizing a protein, said method comprising: disposing said protein in a digestion buffer; disposing a hydrolyzing agent inhibitor in the digestion buffer; passing the digestion buffer comprising said protein and said hydrolyzing agent inhibitor through a reaction chamber comprising at least one hydrolyzing agent, wherein said protein contacts said hydrolyzing agent in the presence of said inhibitor and is present in the chamber for a period of time (t) sufficient to produce protein fragments and provide digestion of said protein in the chamber, wherein the passing of the digestion buffer comprising the protein and the hydrolyzing agent inhibitor through the chamber is done at an adjustable flow rate; and performing multi-segment liquid chromatography tandem mass spectrometry (LC MS/MS) to characterize the protein.

In some embodiments, the protein is denatured to provide a denatured protein before being disposed in the digestion buffer. In some embodiments, said denatured protein is reduced and alkylated before being disposed in the digestion buffer. In some embodiments, said protein is alkylated using N-(2-aminoethyl) maleimide.

In some embodiments, said protein is selected from the group consisting of an antibody, an antibody-like molecule, an antibody light chain, an antibody heavy chain, or biologically active fragments and homologs thereof. In some embodiments, said antibody is a monoclonal antibody (mAb). In some embodiments, said antibody is a therapeutic antibody. In some embodiments, said antibody is a bispecific antibody (bsAb).

In some embodiments, disposing the protein in a digestion buffer comprises disposing the protein in the digestion buffer at a first concentration, wherein said first concentration is about 0.2 micrograms per microliter (µg/µL) or less. In some embodiments, the first concentration is about 0.1 µg/µL or less, optionally wherein the first concentration is about 0.02 µg/µL to about 0.1 µg/µL.

In some embodiments, the hydrolyzing agent inhibitor comprises a protein, a peptide, or a buffer, optionally wherein the hydrolyzing agent inhibitor comprises at least one inhibitor selected from the group consisting of guanidinium chloride, bovine serum albumin (BSA), and a protamine. In some embodiments, the hydrolyzing agent inhibitor comprises a protamine. In some embodiments, the protamine comprises one or more peptides, wherein each of the one or more peptides has a sequence selected from the group consisting of SEQ ID Nos: 8-12.

In some embodiments, disposing the protein in a digestion buffer comprises disposing the protein in the digestion buffer at a first concentration, wherein disposing the hydrolyzing agent inhibitor in the digestion buffer comprises disposing the hydrolyzing agent inhibitor in the digestion buffer at a second concentration, and wherein the second concentration is about the same as or greater than the first concentration. In some embodiments, the second concentration is about 1 times to about 3 times that of the first concentration.

In some embodiments, the protein is contacted with the hydrolyzing agent under acidic and highly chaotropic conditions. In some embodiments, said chaotropic conditions are urea at about 6 to about 9 Molar (M). In some embodiments, said urea is at about 6, 7, or 8 M, optionally about 8 M. In some embodiments, said urea is used at a pH of about 3.0 to about 5.0. In some embodiments, said urea is used at a pH of about 3.5 to about 4.5, optionally at a pH of about 3.9 or 4.0.

In some embodiments, the hydrolyzing agent is a protease. In some embodiments, the protease is selected from the group consisting of aspergillopepsin I, LysN protease (Lys-N), LysC endoproteinase (Lys-C), endoproteinase Asp-N (Asp-N), endoproteinase Glu-C (Glu-C) and outer membrane protein T (OmpT). In some embodiments, the protease is aspergillopepsin I (SEQ ID NO: 1) or a biologically active fragment or homolog thereof.

In some embodiments, said hydrolyzing agent is immobilized. In some embodiments, t ranges from about 0.2 seconds (s) to about 20 s. In some embodiments, t ranges from about 0.2 s to about 3 s, optionally about 0.2 s to about 1 s. In some embodiments, the adjustable flow rate about 50 microliters per minute (µL/min) to about 4.0 µL/min. In some embodiments, the adjustable flow rate is about 0.4 µL/min to about 0.9 µL/min.

In some embodiments, the digested protein fragments range from about 3 kilodaltons (kDa) in mass to about 10 kDa in mass. In some embodiments, said characterization of the protein is selected from the group consisting of sequencing, identifying post-translational modifications (PTM), and identifying a site of a disulfide bond. In some embodiments, said PTMs are selected from the group consisting of pyroglutamic acid formation, oxidation, amidation, deamidation, phosphorylation, methylation, acetylation, and glycosylation.

In some embodiments, characterization data is obtained from said LC MS/MS performed on said protein fragments. In some embodiments, the method is performed in a single LC-MS apparatus. In some embodiments, the method is performed in a single run.

In some embodiments, the characterization data comprise at least 85, 90, 95, or 99% of the protein amino acid sequence. In some embodiments, the characterization data comprise the identity of substantially all of the post-translational modifications of said protein. In some embodiments, the characterization data comprise the location of substantially all of the post-translational modifications of said protein. In some embodiments, a combination of electron transfer dissociation (ETD) and collision-based dissociation techniques (collision activated dissociation (CAD)) and higher energy collisional dissociation (HCD)) tandem mass spectrometry are used to characterize the resulting protein fragments.

In some embodiments, the presently disclosed subject matter provides a method for identifying the site of a disulfide bond in a protein, said method comprising: disposing said protein in a digestion buffer; passing the digestion buffer comprising said protein through a reaction chamber comprising at least one hydrolyzing agent, wherein said protein contacts said hydrolyzing agent and is present in the chamber for a period of time (t) sufficient to produce protein fragments and digestion of said protein occurs in the chamber, wherein the passing of the digestion buffer comprising the protein through the chamber is done at an adjustable flow rate; and performing multi-segment liquid chromatography tandem mass spectrometry (LC MS/MS) to characterize the protein. In some embodiments, the protein is an antibody, optionally a bispecific antibody.

In some embodiments, t is less than about 3 seconds (s), optionally wherein t is about 1 s to about 3 s, further optionally wherein t is about 1.9 s. In some embodiments, said protein is disposed in said digestion buffer at a concentration of about 0.02 µg/µL to about 1 µg/µL, optionally about 0.05 µg/µL. In some embodiments, said protein is disposed in said digestion buffer at a concentration of about 0.1 µg/µL or less. In some embodiments, the method is free of the use of ion-ion proton transfer (IIPT).

Accordingly, it is an object of the presently disclosed subject matter to provide methods for characterizing proteins. This and other objects are achieved in whole or in part by the presently disclosed subject matter. Further, an object of the presently disclosed subject matter having been stated above, other objects and advantages of the presently disclosed subject matter will become apparent to those skilled in the art after a study of the following description, Figures, and Examples.

### BRIEF DESCRIPTION OF THE FIGURES

FIG. 1A is a schematic drawing showing an enzyme reactor of the presently disclosed subject matter and a graph showing the general relationship between protein concentration and the number of enzyme cleavages.

FIG. 1B is a schematic drawing representing bispecific antibody (bsAb) preparation prior to digestion with the enzyme reactor.

FIGS. 2A-2D are a series of graphs showing the total ion current chromatograms for the digestion of apomyoglobin at (A) 0.2 µg/µL, (B) 0.1 µg/µL, (C) 0.05 µg/µL, and (D) 0.02 µg/µL. All digestion times are ~1 second (s). Generally, smaller peptides eluted at ~12-20 minutes while larger peptides eluted at ~25-35 minutes. The star in (A) and (B) denotes undigested apomyoglobin.

FIGS. 3A-3D are a series of graphs showing the total ion current chromatograms for the digestion of protamine treated apomyoglobin at (A) 0.2 µg/µL, (B) 0.1 µg/µL, (C) 0.05 µg/µL, and (D) 0.02 µg/µL. All digestion times are ~1 second (s). The concentration of protamine was 0.2 µg/µL for each sample. The star in (A), (B), and (C) denotes undigested apomyoglobin. In all chromatograms, the first peak at ~12 minutes is attributed to protamine.

FIGS. 4A-4C are a series of graphs showing comparison of selected peptides from the ~1 second (s) digestions of (A) apomyoglobin, (B) β-lactoglobulin, and (C) chicken lysozyme across different concentrations, as well as treatment with 0.2 µg/µL protamine. As concentration decreases in the absence of protamine, the larger peptides decrease in abundance while smaller peptides increase in abundance. Treatment with protamine inhibits the production of smaller peptides while preserving larger peptides. However, at 0.02 µg/µL with protamine, the larger peptides were not recovered at the same abundance as that in the 0.2 and 0.05 µg/µL digestions.

FIGS. 5A and 5B are a series of graphs showing the total ion current chromatograms for the digestion of a bsAb at 0.2 µg/µL (A) without protamine and (B) with protamine. Digest times for both chromatograms were ~1 second (s).

FIGS. 6A and 6B are a pair of graphs comparing peptides observed in the bsAb digestions. More particularly, (A) is a histogram depicting the number of peptides grouped by difference in abundance. (B) is a scatter plot of the log of the ratio of abundance in the treated over untreated by the molecular weight. Points between the grey dashed lines represent peptides that were within 4 times variation.

FIGS. 7A-7E are a series of peptide and composite fragment ion coverage maps of the (A) Common Light Chain (SEQ ID NO: 25), (B) Fd′ A (SEQ ID NO: 26), (C) Fc/2 A (SEQ ID NO: 27), (D) Fd′ B (SEQ ID NO: 28), and (E) Fc/2 B (SEQ ID NO: 29) of a bispecific antibody. Flat hash marks represent identified c and z· fragment ions while angled hash marks represent identified b and y fragment ions. The shaded N61 in maps (C) and (E) represent the localization of a G0F or G1F glycan tree.

FIGS. 8A-8C are a series of total ion current chromatograms for the digestion of chicken lysozyme at (A) 0.2 µg/µL, (B) 0.05 µg/µL, and (C) 0.02 µg/µL. All digestion times were ~1 second (s).

FIGS. 9A-9C are a series of total ion current chromatograms for the digestion of bovine β-lactoglobulin at (A) 0.2 µg/µL, (B) 0.05 µg/µL, and (C) 0.02 µg/µL. All digestion times were ~1 second (s).

FIGS. 10A-10C are a series of total ion current chromatograms for the digestion of bovine trypsinogen at (A) 0.2 µg/µL, (B) 0.05 µg/µL, and (C) 0.02 µg/µL. All digestion times were ~1 second (s).

FIGS. 11A-11C are a series of total ion current chromatograms for the digestion of apomyoglobin at differing concentrations with optimized digestion times to mitigate concentration effects (A) ~1 second (s) digestion at 0.2 µg/µL, (B) ~700 millisecond (ms) digestion at 0.1 µg/µL, and (C) ~400 ms digestion at 0.05 µg/µL.

FIGS. 12A-12C are a series of total ion current chromatograms for the digestion of chicken lysozyme at (A) 0.2 µg/µL, (B) 0.05 µg/µL with 0.2 µg/µL protamine, and (C) 0.02 µg/µL with 0.2 µg/µL protamine. All digestion times were ~1 second (s).

FIGS. 13A-13C are a series of total ion current chromatograms for the digestion of bovine β-lactoglobulin at (A) 0.2 µg/µL, (B) 0.05 µg/µL with 0.2 µg/µL protamine, and (C) 0.02 µg/µL with 0.2 µg/µL protamine. All digestion times were ~1 second (s).

FIG. 14 is a total ion current chromatogram for the ~900 millisecond (ms) digestion of protamine at 0.2 µg/µL. The MS¹ insert to the left of the peak depicts the mass range in which the species elute. The most abundant peaks are undigested protamine. The MS¹ insert to the right of the peak depicts the masses of the peptides produced by the digestion of protamine.

FIGS. 15A and 15B are a pair of total ion current chromatograms for the ~1 second (s) digestion of apomyoglobin with 0.2 µg/µL protamine when rinsed for one hour with (A) 0% organic solvent and (B) 4% organic solvent. The star in (A) denotes the peptide FDKFKHLKTE (SEQ ID NO: 2) from apomyoglobin, which is lost after the 4% rinse.

FIGS. 16A-16B are a pair of total ion current chromatograms for the digestion of bovine trypsinogen at (A) 0.2 µg/µL and (B) 0.05 µg/µL with 0.2 µg/µL protamine. Both digestion times were ~1 second (s). The inserted MS1 shows that the final peak in (B) represents the undigested protein.

FIG. 17 is a schematic showing the principle of size-controlled proteolysis using an enzyme reactor. See also FIG. 18.

FIG. 18 is a diagram of enzyme reactor. The fused silica capillary (i.d. 150 µm) was packed with aldehyde functionalized particles (beads) covalently linked with the protease, aspergillopepsin I (circles). Three measurable values are labeled as L_{packed}, representing the length of the portion packed with protease particles; L_{empty}, representing the length of the empty portion of the column; and V_{water}, representing the total volume of water trapped in the whole column including the portion packed with protease particles. An entry point is provided for the sample which passes through the column at an adjustable flow rate and digestion occurs in the chamber, and an exit point allows for retrieval of digested protein to be used for characterization of protein fragments using techniques such as LC MS/MS.

FIG. 19 is a total ion current chromatogram of a ~1.9 second (s) digestion of a nonreduced bispecific T cell engager (BiTE) antibody.

FIGS. 20A-20E are graphs related to the identification of disulfide peptides of the bispecific T cell engager BiTE antibody including: (A) the total ion chromatogram from 28-34 minutes; (B) the extracted ion chromatogram of the disulfide bound peptides VTMSCKSSQSLLNSGNQKNY (SEQ ID NO: 3) and LAVYYCQNDYSYPLTFGAGTK (SEQ ID NO: 30); (C) the MS¹ taken at 30.5 minutes corresponding to the elution of disulfide bound peptides (SEQ ID NO. 3 and SEQ ID NO: 30). The * labeled peaks show the z=5 and z=4 charge states of the example peptides. FIG. 20D is the higher-energy collisional dissociation (HCD) MS² of the z=5 example peptides (SEQ ID NO: 3 and SEQ ID NO: 30) Observed fragment ions are shown above the spectrum with hash marks corresponding to b- and y-ions. FIG. 20E is the electron transfer dissociation (ETD) MS² of the z=5 example peptides (SEQ ID NO: 3 and SEQ ID NO: 30). The unreacted precursor and reduced species are labeled. The two peaks labeled with arrows represent the cleavage of the disulfide bond resulting in the molecular weights of the individual peptides. Observed fragment ions in the spectrum are shown above the spectrum with hash marks corresponding to c- and z′-ions.

FIG. 21 is a manually annotated peptide map from the nonreduced bispecific T cell engager (BiTE) (SEQ ID NO: 7) digestion.

FIG. 22 is a collisional sequence coverage map obtained from the analysis of a nonreduced bispecific T cell engager (BiTE) (SEQ ID NO: 7). 447/509 residue cleavages were obtained resulting in 88% sequence coverage.

FIG. 23 is an ETD sequence coverage mag obtained from the analysis of a nonreduced bispecific T cell engager (BiTE) (SEQ ID NO: 7). 387/509 residue cleavages were obtained resulting in 76% sequence coverage. Coverage by ETD suffers in the ~200-275 region due to a lack of basic residues. This leads to charge depleted peptides which do not dissociate well by ETD.

FIG. 24 is a composite sequence coverage map obtained by collisional fragmentation and ETD from the analysis of a nonreduced bispecific T cell engager (BiTE) (SEQ ID NO: 7). 499/509 residue cleavages were obtained resulting in 98% sequence coverage. Flat hash marks represent identified ETD fragment ions while angled hash marks represent identified collisional (HCD and CAD) fragment ions. All 12 complementary determining regions (CDRs) are underlined.

FIG. 25 is a schematic diagram showing the disulfide bonds of a nonreduced bispecific T cell engager (BiTE) antibody identified by the presently disclosed method. A disulfide bonded pair was present in each region of the BiTE. The linker sequences (GGGGSGGGGSGGGGS, SEQ ID NO: 4; GGGGS, SEQ ID NO: 5; and SGGSGGSGG, SEQ ID NO: 6) are noted between each region.

FIGS. 26A-26D are a series of graphs of the chromatogram of tracked peptides from digestions of apomyoglobin including (A) the total ion current of a ~1 second digestion of 0.2 microgram per microliter (µg/µL) apomyoglobin; (B) the extracted ion chromatogram of 12 peptides. The numbers represent the Peptide # given in Table 4. Since this digestion is closer to a catalytic limited system, all 12 peptides should be present when apomyoglobin is digested under these conditions. (C) is a graph of the total ion current of a ~1 second digestion of 0.05 µg/µL apomyoglobin; and (D) is a graph of the extracted ion chromatogram of the same 12 peptides as FIG. 26B. Only 3 of the 12 peptides are present in this run due to the diffusion limited system.

FIGS. 27A and 27B are graphs showing the chromatograms of ubiquitin treated Apomyoglobin digestion, where (A) is the total ion current chromatogram of a ~1 second digestion of 0.15 microgram per microliter (µg/µL) ubiquitin + 0.05 µg/µL apomyoglobin; and (B) is an extracted ion chromatogram of the 12 tracked peptides. Only 3 of the 12 possible peptides were observed, which implies that apomyoglobin is still in a diffusion limited system.

FIGS. 28A and 28B are chromatograms of β-Lactoglobulin treated Apomyoglobin digestion, where (A) is the total ion current chromatogram of a ~1 second digestion of 0.15 microgram per microliter (µg/µL) β-Lactoglobulin + 0.05 µg/µL apomyoglobin; and (B) is the extracted ion chromatogram of the 12 tracked peptides. Only 4 of the 12 possible peptides were observed, which implies that apomyoglobin is still in a diffusion limited system.

FIGS. 29A and 29B are chromatograms of bovine serum albumin (BSA) treated Apomyoglobin digestion, where (A) is the total ion current chromatogram of a ~1 second digestion of 0.15 micrograms per microliter (µg/µL) BSA + 0.05 µg/µL apomyoglobin; and (B) is the extracted ion chromatogram of the 12 tracked peptides. 10 of the 12 possible peptides were observed, which implies that apomyoglobin was successfully inhibited. However, many of the apomyoglobin peaks were suppressed by peptides from BSA.

### DETAILED DESCRIPTION

Recent interest in bispecific antibodies promoted the development of fast and accurate methods of analysis to ensure the molecule is correctly produced. Unlike monospecific antibodies, each subunit of a bispecific antibody has a different primary structure, effectively halving the concentration of each respective subunit. Presented herein is the characterization of the effects of protein concentration on digestion using an immobilized aspergillopepsin I enzyme reactor. This reproducible, nonspecific enzyme efficiently digests a protein in hundreds of milliseconds, providing complete sample preparation in a few hours. Four exemplary proteins were digested using a constant digestion time at varying concentrations to characterize the resulting peptide profile as a control. Low concentration samples resulted in diffusion limited digestions with elimination of large peptide products due to a greater number of enzymatic cleavages. A competitive inhibitor was used to reduce the number of enzymatic cleavages to the protein and to retain large molecular weight products. In particular, one exemplary competitive inhibitor used herein was protamine, which is a highly basic molecule comprising primarily arginine. When this exemplary inhibitor was used on a bispecific antibody, complete sequence coverage was successfully obtained in a single chromatographic analysis. Methods involving lower concentration samples also resulted in localization of disulfide bonds.

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

### I. Definitions

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. Mention of techniques employed herein are intended to refer to the techniques as commonly understood in the art, including variations on those techniques or substitutions of equivalent techniques that would be apparent to one of skill in the art. Thus, unless defined otherwise, all technical and scientific terms and any acronyms used herein have the same meanings as commonly understood by one of ordinary skill in the art in the field of the presently disclosed subject matter. Although any compositions, methods, kits, and means for communicating information similar or equivalent to those described herein can be used to practice the presently disclosed subject matter, particular compositions, methods, kits, and means for communicating information are described herein. It is understood that the particular compositions, methods, kits, and means for communicating information described herein are exemplary only and the presently disclosed subject matter is not intended to be limited to just those embodiments.

Following long-standing patent law convention, the terms “a”, “an”, and “the” refer to “one or more” when used in this application, including the claims. Thus, in some embodiments the phrase “a peptide” refers to one or more peptides.

As used herein, the term “and/or” when used in the context of a list of entities, refers to the entities being present singly or in any and every possible combination and subcombination. Thus, for example, the phrase “A, B, C, and/or D” includes A, B, C, and D individually, but also includes any and all combinations and subcombinations of A, B, C, and D. It is further understood that for each instance wherein multiple possible options are listed for a given element (i.e., for all “Markush Groups” and similar listings of optional components for any element), in some embodiments the optional components can be present singly or in any combination or subcombination of the optional components. It is implicit in these forms of lists that each and every combination and subcombination is envisioned and that each such combination or subcombination has not been listed simply merely for convenience. Additionally, it is further understood that all recitations of “or” are to be interpreted as “and/or” unless the context clearly requires that listed components be considered only in the alternative (e.g., if the components would be mutually exclusive in a given context and/or could not be employed in combination with each other).

The term “about,” as well as the symbol “~,” as used herein, mean approximately, in the region of, roughly, or around. When the term “about” or the symbol “~” is used in conjunction with a numerical range, it modifies that range by extending the boundaries above and below the numerical values set forth. In general, the term “about” or the symbol “~” is used herein to modify a numerical value above and below the stated value by a variance of 10%. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” or the symbol “~.”

As used herein the term, “accurate mass” refers to an experimentally or theoretically determined mass of an ion that is used to determine an elemental formula. For ions containing combinations of the elements C, H, N, O, P, S, and the halogens, with mass less than 200 Unified Atomic Mass Units, a measurement about 5 ppm uncertainty is sufficient to uniquely determine the elemental composition.

As used herein, amino acids are represented by the full name thereof, by the three letter code corresponding thereto, or by the one-letter code corresponding thereto, as indicated in the following table:

| | | | |
|---|---|---|---|
| Full Name | 3-Letter Code | 1-Letter Code | Functionally Equivalent Codons |
| Aspartic Acid | Asp | D | GAC GAU |
| Glutamic Acid | Glu | E | GAA GAG |
| Lysine | Lys | K | AAA AAG |
| Arginine | Arg | R | AGA AGG CGA CGC CGGCGU |
| Histidine | His | H | CAC CAU |
| Tyrosine | Tyr | Y | UAC UAU |
| Cysteine | Cys | C | UGC UGU |
| Asparagine | Asn | N | AAC AAU |
| Glutamine | Gln | Q | CAA CAG |
| Serine | Ser | S | ACG AGU UCA UCC UCG UCU |
| Threonine | Thr | T | ACA ACC ACG ACU |
| Glycine | Gly | G | GGA GGC GGG GGU |
| Alanine | Ala | A | GCA GCC GCG GCU |
| Valine | Val | V | GUA GUC GUG GUU |
| Leucine | Leu | L | UUA UUG CUA CUC CUGCUU |
| Isoleucine | Ile | I | AUA AUC AUU |
| Methionine | Met | M | AUG |
| Proline | Pro | P | CCA CCC CCG CCU |
| Phenylalanine | Phe | F | UUC UUU |
| Tryptophan | Trp | W | UGG |

The term “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the presently disclosed subject matter, and particularly at the carboxy- or amino-terminus, can be modified by methylation, amidation, acetylation or substitution with other chemical groups which can change the peptide’s circulating half-life without adversely affecting their activity. Additionally, a disulfide linkage can be present or absent in the peptides of the presently disclosed subject matter.

The term “amino acid” is used interchangeably with “amino acid residue,” and can refer to a free amino acid and to an amino acid residue of a peptide. It will be apparent from the context in which the term is used whether it refers to a free amino acid or a residue of a peptide.

Amino acids have the following general structure: H₂N—C(H)(R)—COOH. Amino acids can be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino- and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e.g., 5-fluorouracil is an analog of thymine).

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the presently disclosed subject matter can exist in a variety of forms including, for example, polyclonal antibodies, monoclonal antibodies, Fv, Fab and F(ab)₂, as well as single chain antibodies and humanized antibodies.

An “antibody heavy chain,” as used herein, refers to the larger of the two types of polypeptide chains present in naturally occurring antibody molecules.

An “antibody light chain,” as used herein, refers to the smaller of the two types of polypeptide chains present in naturally occurring antibody molecules.

By the term “synthetic antibody” as used herein, is meant an antibody which is generated using recombinant DNA technology or other approach for engineering antibodies, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “biological sample,” as used herein, refers to samples obtained from a subject, including, but not limited to, skin, hair, tissue, blood, plasma, cells, sweat and urine.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner,” as used herein, refers to a molecule capable of binding to another molecule.

As used herein, the term “biologically active fragments” or “bioactive fragment” of the polypeptides encompasses natural or synthetic portions of the full-length protein that are capable of specific binding to their natural ligand or of performing the function of the protein.

A “chaotropic agent” is a substance which disrupts the structure of, and denatures, macromolecules such as proteins and nucleic acids (e.g. DNA and RNA). Chaotropic solutes increase the entropy of the system by interfering with intramolecular interactions mediated by non-covalent forces such as hydrogen bonds, van der Waals forces, and hydrophobic effects. Macromolecular structure and function is dependent on the net effect of these forces (see protein folding), therefore it follows that an increase in chaotropic solutes in a biological system will denature macromolecules, reduce enzymatic activity and induce stress on a cell (i.e., a cell will have to synthesize stress protectants). Tertiary protein folding is dependent on hydrophobic forces from amino acids throughout the sequence of the protein. Chaotropic solutes decrease the net hydrophobic effect of hydrophobic regions because of a disordering of water molecules adjacent to the protein. This solubilizes the hydrophobic region in the solution, thereby denaturing the protein. This is also directly applicable to the hydrophobic region in lipid bilayers; if a critical concentration of a chaotropic solute is reached (in the hydrophobic region of the bilayer) then membrane integrity will be compromised, and the cell will lyse. Chaotropic salts that dissociate in solution exert chaotropic effects via different mechanisms. Whereas chaotropic compounds such as ethanol interfere with non-covalent intramolecular forces as outlined above, salts can have chaotropic properties by shielding charges and preventing the stabilization of salt bridges. Hydrogen bonding is stronger in non-polar media, so salts, which increase the chemical polarity of the solvent, can also destabilize hydrogen bonding. Mechanistically this is because there are insufficient water molecules to effectively solvate the ions. This can result in ion-dipole interactions between the salts and hydrogen bonding species which are more favorable than normal hydrogen bonds. Chaotropic agents include butanol, ethanol, guanidinium chloride, lithium perchlorate, lithium acetate, magnesium chloride, phenol, propanol, sodium dodecyl sulfate, thiourea and urea.

As used herein, the term “chemically conjugated,” or “conjugating chemically” refers to linking the antigen to the carrier molecule. This linking can occur on the genetic level using recombinant technology, wherein a hybrid protein can be produced containing the amino acid sequences, or portions thereof, of both the antigen and the carrier molecule. This hybrid protein is produced by an oligonucleotide sequence encoding both the antigen and the carrier molecule, or portions thereof. This linking also includes covalent bonds created between the antigen and the carrier protein using other chemical reactions, such as, but not limited to glutaraldehyde reactions. Covalent bonds can also be created using a third molecule bridging the antigen to the carrier molecule. These cross-linkers are able to react with groups, such as but not limited to, primary amines, sulfhydryls, carbonyls, carbohydrates or carboxylic acids, on the antigen and the carrier molecule. Chemical conjugation also includes non-covalent linkage between the antigen and the carrier molecule.

The term “competitive sequence” refers to a peptide or a modification, fragment, derivative, or homolog thereof that competes with another peptide for its cognate binding site.

A “compound,” as used herein, refers to any type of substance or agent that is commonly considered a drug, or a candidate for use as a drug, as well as combinations and mixtures of the above.

As used herein, the term “conservative amino acid substitution” is defined herein as an amino acid exchange within one of the following five groups:
I. Small aliphatic, nonpolar or slightly polar residues: Ala, Ser, Thr, Pro, Gly;
II. Polar, negatively charged residues and their amides: Asp, Asn, Glu, Gln;
III. Polar, positively charged residues: His, Arg, Lys;
IV. Large, aliphatic, nonpolar residues: Met Leu, Ile, Val, Cys; and
V. Large, aromatic residues: Phe, Tyr, Trp.

As used herein, a “derivative” of a compound, when referring to a chemical compound, is one that can be produced from another compound of similar structure in one or more steps, as in replacement of H by an alkyl, acyl, or amino group.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

As used herein, the term “domain” refers to a part of a molecule or structure that shares common physicochemical features, such as, but not limited to, hydrophobic, polar, globular and helical domains or properties such as ligand binding, signal transduction, cell penetration and the like. Specific examples of binding domains include, but are not limited to, DNA binding domains and ATP binding domains. As used herein, the term “effector domain” refers to a domain capable of directly interacting with an effector molecule, chemical, or structure in the cytoplasm which is capable of regulating a biochemical pathway.

By “equivalent fragment” as used herein when referring to two homologous proteins from different species is meant a fragment comprising the domain or amino acid being described or compared relative to the first protein.

As used herein, an “essentially pure” preparation of a particular protein or peptide is a preparation wherein at least about 95%, and preferably at least about 99%, by weight, of the protein or peptide in the preparation is the particular protein or peptide.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, the term “fragment,” as applied to a protein or peptide, can ordinarily be at least about 2-15 amino acids in length, at least about 15-25 amino acids, at least about 25-50 amino acids in length, at least about 50-75 amino acids in length, at least about 75-100 amino acids in length, and greater than 100 amino acids in length, depending on the particular protein or peptide being referred to.

As used herein, a “functional” molecule is a molecule in a form in which it exhibits a property or activity by which it is characterized. A functional enzyme, for example, is one that exhibits the characteristic catalytic activity by which the enzyme is characterized.

“Highly chaotropic environment” refers the concentration of a chaotropic agent in a solution. In some embodiments, the concentration is exactly, about or at least, 1, 2, 3, 4, 5, 6, 7, 8, 9, 10, or more molar. In some embodiments, it refers to about or at least 6, 7, 8 or 9 molar urea.

As used herein, “homology” is used synonymously with “identity.” The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used. The percent identity between two sequences can be determined using techniques similar to those described above, with or without allowing gaps. In calculating percent identity, typically exact matches are counted.

As used herein, the term “hydrolyzing agent” refers to any one or combination of a large number of different enzymes, including but not limited to aspergillopepsin I, trypsin, Lysine-C endopeptidase (LysC), arginine-C endopeptidase (ArgC), Asp-N, glutamic acid endopeptidase (GluC) and chymotrypsin, V8 protease and the like, as well as chemicals, such as cyanogen bromide. In the presently disclosed subject matter, one or a combination of hydrolyzing agents cleave peptide bonds in a protein or polypeptide, in a sequence-specific manner or a non-sequence-specific manner, generating a collection of shorter peptides (a “digest”). A portion of the biological samples are contacted with hydrolyzing agent(s) to form a digest of the biological sample. Given that the amino acid sequences of certain polypeptides and proteins in biological samples are often known and that the hydrolyzing agent(s) cuts in a sequence-specific manner, the shorter peptides in the digest are generally of a predicable amino acid sequence.

As used herein, an “instructional material” includes a publication, a recording, a diagram, or any other medium of expression which can be used to communicate the usefulness of the compositions and apparatuses of the presently disclosed subject matter in the kit. The instructional material of the kit of the presently disclosed subject matter can, for example, be affixed to a container which contains the identified compound(s) of the presently disclosed subject matter or be shipped together with a container which contains the identified compound(s). Alternatively, the instructional material can be shipped separately from the container with the intention that the instructional material and the compound be used cooperatively by the recipient.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, e.g., through ionic or hydrogen bonds or van der Waals interactions.

“Liquid chromatography-mass spectrometry (LC-MS, or alternatively HPLC-MS)” is an analytical chemistry technique that combines the physical separation capabilities of liquid chromatography (or HPLC) with the mass analysis capabilities of mass spectrometry (MS). Liquid chromatography generally utilizes very small particles packed and operating at relatively high pressure and is referred to as high performance liquid chromatography (HPLC). LC-MS methods use HPLC instrumentation for sample introduction. In HPLC, the sample is forced by a liquid at high pressure (the mobile phase) through a column that is packed with a stationary phase generally composed of irregularly or spherically shaped particles chosen or derivatized to accomplish particular types of separations. HPLC methods are historically divided into two different sub-classes based on stationary phases and the corresponding required polarity of the mobile phase. Use of octadecylsilyl (C18) and related organic-modified particles as stationary phase with pure or pH-adjusted water-organic mixtures such as water-acetonitrile and water-methanol are used in techniques termed reversed phase liquid chromatography (RP-LC). Use of materials such as silica gel as stationary phase with neat or mixed organic mixtures are used in techniques termed normal phase liquid chromatography (NP-LC).

The term “mass spectrometer” means a device capable of detecting specific molecular species and measuring their accurate masses. The term is meant to include any molecular detector into which a polypeptide or peptide can be eluted for detection and/or characterization. In the preferred MS procedure, a sample, e.g., the elution solution, is loaded onto the MS instrument, and undergoes vaporization. The components of the sample are ionized by one of a variety of methods (e.g., by electrospray ionization or “ESI”), which results in the formation of positively charged particles (ions). The positive ions are then accelerated by a magnetic field. The computation of the mass-to-charge ratio of the particles is based on the details of motion of the ions as they transit through electromagnetic fields, and detection of the ions. In some embodiments, the mass measurement error of a mass spectrometer is about 10 ppm or less, in another it is about 7 ppm or less, and in yet another it is about 5 ppm or less. Fragment ions in the MS/MS (MS²) and MS³ spectra are generally highly specific for peptides of interest.

The term “peptide” typically refers to short polypeptides.

The term “per application” as used herein refers to administration of a compositions, drug, or compound to a subject.

“Plurality” means at least two.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross and Mienhofer, eds., The Peptides, vol. 3, pp. 3-88 (Academic Press, New York, 1981) for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

The support can be either biological in nature, such as, without limitation, a cell or bacteriophage particle, or synthetic, such as, without limitation, an acrylamide derivative, agarose, cellulose, nylon, silica, or magnetized particles.

By the term “specifically binds to”, as used herein, is meant when a compound or ligand functions in a binding reaction or assay conditions which is determinative of the presence of the compound in a sample of heterogeneous compounds.

The term “standard,” as used herein, refers to something used for comparison. For example, a standard can be a known standard agent or compound which is administered or added to a control sample and used for comparing results when measuring said compound in a test sample. In some embodiments, the standard compound is added or prepared at an amount or concentration that is equivalent to a normal value for that compound in a normal subject. Standard can also refer to an “internal standard,” such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

As used herein, a “substantially homologous amino acid sequence” includes those amino acid sequences which have at least about 95% homology, preferably at least about 96% homology, more preferably at least about 97% homology, even more preferably at least about 98% homology, and most preferably at least about 99% homology to an amino acid sequence of a reference sequence. Amino acid sequences similarity or identity can be computed using, for example, the BLASTP and TBLASTN programs which employ the BLAST (basic local alignment search tool) algorithm. The default setting used for these programs are suitable for identifying substantially similar amino acid sequences for purposes of the presently disclosed subject matter.

“Substantially identical” when referring to a subject protein or polypeptide relative to a reference protein or polypeptide (e.g., an enzyme such as aspergillopepsin I or a enzymatically active fragment thereof) means that the subject is either exactly, at least or about 99.9, 99.5, 99, 98, 97, 96, 95, 94, 93, 92, 91, 90, 89, 88, 87, 86, 85, 84, 83, 82, 81, 80, 75, 70, 65 or 60 percent identical in terms of amino acid sequence relative to the reference.

The term “substantially pure” describes a compound, e.g., a protein or polypeptide which has been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

### II. General Considerations

The demands for characterization of therapeutic antibodies are increasing with the rapid development of antibody-based pharmaceuticals. MS is one of the most powerful techniques for the structural characterization of therapeutic antibodies due to its high accuracy, resolution, and speed over other analytical techniques.

The implementation of an immobilized aspergillopepsin I enzyme reactor to digest monospecific antibodies and analyze them in a single LC/MS/MS analysis has been implemented.^{12,13} See also, U.S. Pat. No. 10,281,473, incorporated herein by reference in its entirety. Aspergillopepsin I (SEQ ID NO:1) is an aspartic protease that functions in 8 M urea and acidic conditions. While the enzyme tends to cleave around hydrophobic residues as well as C-terminal to lysines, the enzyme is considered nonspecific since it is capable of cleaving between any two amino acids.¹⁴ Although the enzyme is nonspecific, the resulting digestion is highly reproducible given the same working conditions (i.e. digestion time, sample concentration, buffer conditions, etc.). However, in-solution digestions are not feasible due to the rapid nature of Aspergillopepsin I. Therefore, the digestion is controlled by immobilizing the enzyme to beads. Proteins are digested by flowing over the enzyme-bound beads packed into a column so that the digestion stops once the products elute from the column. Tailoring the flow rate through the column dictates the peptide size distribution; as flow rate increases, the interaction time between enzyme and protein decreases resulting in fewer cleavages.¹² Since Aspergillopepsin I is a fast enzyme, digestions are performed on the timescale of hundreds of milliseconds. Digested proteins are fully characterized from a single LC-MS/MS analysis due to the extraordinary number of peptides generated from the single digestion, resulting in drastically reduced preparation and analysis time. Within this single analysis, multiple overlapping peptides are observed to provide unambiguous sequence coverage of a molecule.¹³ SEQ ID NO:1 (aspergillopepsin I)-

MVVFSKTAALVLGLSTAVSAAPAPTRKGFTINQIARPANKTRTVNLPGLY
ARSLAKFGGTVPQSVKEAASKGSAVTTPQNNDEEYLTPVTVGKSTLHLDF
DTGSADLWVFSDELPSSEQTGHDLYTPSSSATKLSGYSWDISYGDGSSAS
GDVYRDTVTVGGVTTNKQAVEAASKISSEFVQDTANDGLLGLAFSSINTV
QPKAQTTFFDTVKSQLDSPLFAVQLKHDAPGVYDFGYIDDSKYTGSITYT
DADSSQGYWGFSTDGYSIGDGSSSSSGFSAIADTGTTLILLDDEIVSAYY
EQVSGAQESYEAGGYVFSCSTDLPDFTVVIGDYKAVVPGKYINYAPVSTG
SSTCYGGIQSNSGLGLSILGDVFLKSQYVVFNSEGPKLGFAAQA

In some embodiments, the presently disclosed subject matter addresses the concentration of the sample prior to digestion and the impacts of the changes on the resulting digestion profile. In some embodiments, at least two factors can affect desired sample flow rate, IgG concentration (or other test protein) and the length of the packed reactor. To produce peptides with a desired size range (i.e. medium size 3 kDa to 10 kDa) using a given enzyme reactor column, lower protein concentration requires a high flow rate (i.e. less digestion time). But, additional considerations of the effects of concentration need to be brought to bear to provide improved performance, in accordance with the presently disclosed subject matter. The presently disclosed subject matter exemplifies additional concentration effects, in the data and figures, and advantageously employs the concentration effects.

In some embodiments, the presently disclosed subject matter employs a faster digestion rate from a lower concentration to advantageously localize disulfide bonds. For example, by decreasing the concentration of the sample, significantly lower digestion times (by way of example but not limitation, ~3 seconds (s) or less) can obtain disulfide bond localization. Also, in some embodiments, localization of the disulfide bonds can be accomplished with a more accessible mass spectrometry method that uses both electron transfer dissociation (ETD) and collision-based techniques. While ion-ion proton transfer (IIPT) can be used as well, it is not required in the localization of disulfide bonds.

In some embodiments, the presently disclosed subject matter provides for the use of an inhibitor of the hydrolyzing agent, wherein the protein to be characterized contacts the hydrolyzing agent in the presence of said inhibitor. The inhibitor mitigates the effect of the lower concentrations of the protein, which is useful because not all samples are ideally concentrated. In some embodiments, protamines are employed as the inhibitor additive to mitigate these affects. However, other inhibitors can be used to slow down the reaction rate, such as but not limited to, another protein or peptide, and/or a buffer. In some embodiments, the hydrolyzing agent inhibitor comprises at least one inhibitor selected from the group consisting of guanidinium chloride, bovine serum albumin (BSA), and a protamine. The inhibitors have an effect on reaction rate. For example, the protamine inhibitor was shown to be useful in increasing the sequence coverage of bispecific antibodies, whose different segments have different concentrations under the conditions used for the reactor.

By way of elaboration and not limitation, in some embodiments, a micro-column containing an immobilized proteinase, aspergillopepsin 1, is employed at flow rates of proteins in 8 M urea on the order of seconds to generate large pieces of proteins that are then sequenced by tandem mass spectrometry. Despite how well this works, it was found that many of the large protein fragments failed to quickly diffuse away from the beads and thus often underwent additional cleavage reactions to produce undesirable smaller fragments. In accordance with the presently disclosed subject matter, the multi-step cleavage of protein large fragments can be suppressed by adding a competitive inhibitor, e.g., a protamine, (e.g., MPRRRRSSSR PVRRRRRPRV SRRRRRRGGR RRR, SEQ ID NO: 8) to the protein digest. Under the new conditions, adjacent enzyme sites are now occupied with digestion of the protamine (aspergillopepsin prefers to cleave after K, R, and small hydrophobic residues) and unavailable for further digestion of protein fragments. The result is enhanced production of large protein fragments that allow complete, or near complete, sequence coverage of important biological proteins such as antibodies.

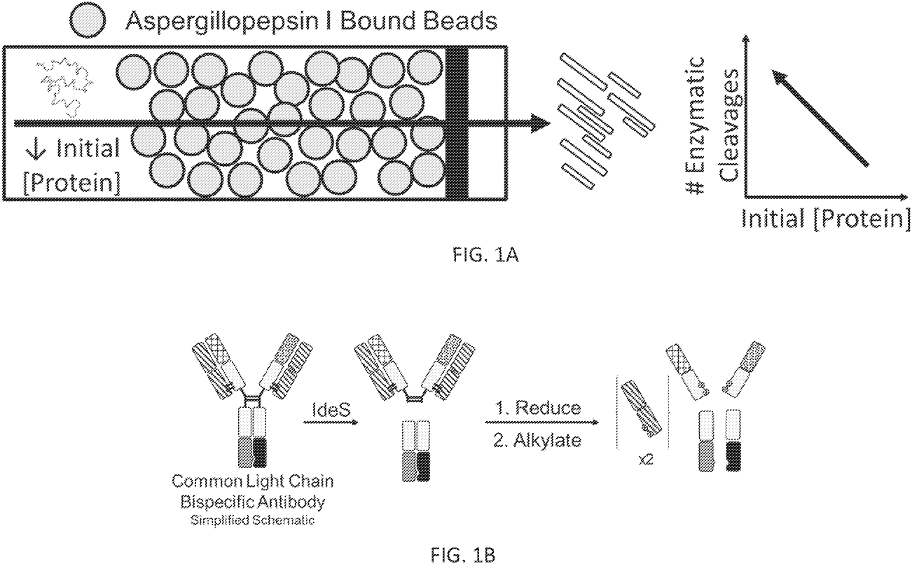

More particularly, the presently disclosed subject matter describes the exemplary analysis of an IgG-like bsAb with a common light chain.¹⁵ This molecule retains the ~150 kDa structure of a monospecific IgG antibody (i.e. two heavy chains and two light chains). However, each heavy chain has a different variable region as well as a knob-in-hole mutation in the fragment, crystallizable (Fc/2) region.^{16,17} The same light chain is paired to each heavy chain, which drastically increases the generation of the proper antibody configuration.¹⁸ Since the heavy chains are different, their relative concentration is half compared to a monospecific antibody after reduction and alkylation. This drop in concentration presents a challenge to control the rate of digestion by an immobilized nonspecific enzyme reactor. To understand the implications of the varying chain concentrations, the studies described herein use standard proteins to investigate the effect of protein concentration on the rate of digestion using the aspergillopepsin I enzyme reactor. See FIG. 1A. An approach to mitigate these concentration dependent effects is demonstrated and applied to a bispecific antibody with a common light chain.

### III. Representative Embodiments

In some embodiments, the presently disclosed subject matter provides a method of characterizing a protein in a sample. In some embodiments, the method comprises: optionally denaturing the protein; disposing said protein in a digestion buffer; passing the digestion buffer comprising said protein through a reaction chamber comprising at least one hydrolyzing agent and an inhibitor of the hydrolyzing agent, wherein said protein contacts said hydrolyzing agent in the presence of said inhibitor and is present in the chamber for a period of time (t) sufficient to produce protein fragments and digestion of said protein occurs in the chamber, wherein the passing of the digestion buffer comprising the protein through the chamber is done at an adjustable flow rate; and performing multi-segment liquid chromatography tandem mass spectrometry (LC MS/MS) to characterize the protein. In some embodiments, the inhibitor is disposed in the digestion buffer.

In some embodiments, the presently disclosed subject matter provides a method for identifying the site of a disulfide bond in a protein. In some embodiments, the method comprising: disposing said protein in a digestion buffer; passing the digestion buffer comprising said protein through a reaction chamber comprising at least one hydrolyzing agent, wherein said protein contacts said hydrolyzing agent and is present in the chamber for a period of time (t) sufficient to produce protein fragments and digestion of said protein occurs in the chamber, wherein the passing of the digestion buffer comprising the protein through the chamber is done at an adjustable flow rate; and performing multi-segment liquid chromatography tandem mass spectrometry (LC MS/MS) to characterize the protein.

Sample proteins, e.g. antibodies and/or antibody like molecules or other proteins, which are suitable for analysis in the methods of the presently disclosed subject matter include those which are about or less than 500, 400, 300, 200, 150, 100, 75, 50, 25, 10 or 5 kDa in mass. In some embodiments, the protein is a membrane protein. In some embodiments, the protein is an antibody.

The basic antibody structural unit is known to comprise a tetramer. Each tetramer is composed of two identical pairs of polypeptide chains, each pair having one “light” (about 25 kDa) and one “heavy” chain (about 50 kDa -70 kDa). The carboxy-terminal portion of each chain preferably defines a constant region primarily responsible for effector function. Human light chains are classified as kappa and lambda light chains. Heavy chains are classified as mu, delta, gamma, alpha, or epsilon, and define the antibody’s isotype as IgM, IgD, IgG, IgA, and IgE, respectively. See generally, Fundamental Immunology Ch. 7 (Paul, W., ed., 2nd ed. Raven Press, N.Y. (1989)) (incorporated by reference in its entirety for all purposes). The variable regions of each light (“VL”)/heavy chain (“VH”) pair preferably form the antibody binding site. Thus, an intact IgG antibody has two binding sites. Except in bifunctional or bispecific antibodies, the two binding sites are the same. The chains all exhibit the same general structure of relatively conserved framework regions (FR) joined by three hyper variable regions, also called complementarity determining regions or CDRs. The CDRs from the heavy and the light chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)), or Chothia & Lesk, J. Mol. Biol. 196:901-917 (1987); Chothia et al., Nature 342:878-883 (1989).

Examples of molecules which are described by the term “antibody” herein include, but are not limited to: single chain Fvs (scFvs), Fab fragments, Fab′ fragments, F(ab′) 2, disulfide linked Fvs (sdFvs), Fvs, and fragments thereof comprising or alternatively consisting of, either a VL or a VH domain. The term “single chain Fv” or “scFv” as used herein refers to a polypeptide comprising a VL domain of antibody linked to a VH domain of an antibody. As such, the term antibody encompasses not only whole antibody molecules, but also antibody multimers and antibody fragments, as well as variants (including derivatives) of antibodies, antibody multimers, and antibody fragments. Included in the term are T cell receptors, single chain Fvs (scFvs), Fab fragments, Fab′ fragments, F(ab′) 2, disulfide linked Fvs (sdFvs), Fvs, and fragments thereof. One of ordinary skill in the art will appreciate that the compositions and methods of the presently disclosed subject matter can be easily be applied to the characterization of antibody drug conjugates, (ADCs), antibody biosimilars, chimeric antigen receptors (CARs), and antigen T-cell receptors.

In some embodiments, the protein is first denatured. In some embodiments, the denatured protein is reduced and alkylated. In some embodiments, the denatured protein is passed through an enzyme reaction chamber, also referred to herein as an enzyme reactor or reaction chamber for a selected period of digestion time, in order for the protein to be exposed in a time-controlled manner to the hydrolyzing agent.

Digestion times can vary depending on several conditions and parameters and whether a disulfide analysis is to be performed. Digestions times for sequencing can include, for example, about 0.2 s to about 5.7 s, including 0.7 s. In some embodiments, digestion times range from about 0.2 seconds to about 10 minutes. In some embodiments, digestion times range from about 1 s to about 3 s. In some embodiments, digestion times are about 1 s or less. In some embodiments, digestion times are about 200 milliseconds to about 600 milliseconds, including 400 milliseconds. Digestion times for disulfide analysis are longer when the same protein is to be analyzed and can be, for example, 1.9 s, 12 s, 93 s, 260 s, and 740 s. One of ordinary skill in the art can determine the digestion times (times in the column/reactor) based on factors such as the protein being characterized, the hydrolyzing agent being used, the inhibitor being used, the buffer being used, the chaotropic agent being used, the length and diameter of the column/chamber and can use the three Equations provided herein to aid in the process. In some embodiments, the protein is disposed in the digestion buffer at a concentration of about 0.2 micrograms per microliter (µg/µL) or less, optionally of about 0.1 micrograms per microliter (µg/µL) or less. In some embodiments, the protein is disposed in said digestion buffer at a concentration of about 0.02 µg/µL to about 1 µg/µL, optionally about 0.05 µg/µL. In some embodiments, t is less than about 3 seconds (s), optionally wherein t is about 1 s to about 3 s, further optionally wherein t is about 1.9 s. In some embodiments, the method is free of the use of ion-ion proton transfer (IIPT).

In some embodiments, the protein is exposed to the hydrolyzing agent under acidic and highly chaotropic conditions to obtain peptides (fragments) from the protein. Then mass spectroscopy is performed on the peptides to obtain characterization data. In some embodiments, the method is performed in a single LC-MS apparatus. In some embodiments, the method is performed in a single run. In some embodiments, the characterization data comprises at least 85, 90, 95, 99% of the protein amino acid sequence. In some embodiments, the characterization data comprises the identity and/or location of substantially all of the protein’s post-translational modifications.

In some embodiments, the protein is selected from the group comprising an antibody, an antibody-like molecule, an antibody light chain, and antibody heavy chain, or biologically active fragments and homologs thereof. In some embodiments, the antibody is a monoclonal antibody. In some embodiments, the antibody is a bispecific antibody.

In some embodiments, the hydrolyzing agent is aspergillopepsin I enzyme or a biologically active fragment or homolog thereof, or a substantially identical enzyme having aspergillopepsin I enzyme activity.

In some embodiments, the time of passage through the column is about, at least or exactly 1, 2, 3, 5, 6, 7, 8, 9, 10 milliseconds, seconds or minutes.

In some embodiments, the highly chaotropic conditions include about 6 to about 9 Molar urea. In some embodiments, it includes at least or about 6, 7, or 8 Molar urea. In some embodiments, the condition comprises 8 M urea.

As a “compromise” between bottom-up and top-down approaches, “middle-down” analysis has drawn increasing interest. This concept inherits some of the advantages of intact protein MS analysis, but has lower instrumental requirements (e.g. sensitivity, resolution) in achieving sufficient signal-to-noise ratio (S/N) of fragment ions for protein sequencing. Middle-down protein analysis typically involves protein digestion using proteases or chemicals that cleave proteins at single type of amino acid residue to generate peptides generally larger than 3 kDa. Frequently used tools include Lys-C (cleaves at C-terminal size of Lys), Asp-N (cleaves at N-terminal side of Asp), and Glu-C (cleaves at C-terminal side of Glu). High concentrations of formic acid and acetic acid with assistance of microwave radiation have also been employed to cleave C-terminal side of Asp. Some dibasic-site specific proteases are also reported to create even larger peptides (Tsybin et al., J. Proteome Res. 2013, 12, 5558-5569).

Compared to small tryptic peptides, medium or large peptides generally reveal more information of protein isoforms, variants, and combinatorial PTMs. They have fewer source protein candidates in protein databases, leading to higher protein identification confidence by database search. In some embodiments of MS analysis, larger peptides tend to have a higher number of basic amino acid residues, which facilitates peptide sequencing by ETD or ECD. Recent studies have shown the power of middle-down approach in characterization of histone PTMs as well as other proteins.

However, the limitations of currently available tools for middle-down protein analysis are also substantial. For example, none of the twenty amino acids are evenly distributed along protein chains. Protein digestion at single-type amino acid sites still produces many small (<3000 Da) or ultra large (>15 kDa) peptides. Identification/characterization of proteins based on these peptides cannot take advantage of middle-down approach (Tsybin et al., J. Proteome Res., 2013, 12, 5558-5569). Additionally, the enzymatic digestion efficiency is often low for proteins with highly folded structure or low solubility.

Although high concentrations of chaotropic agents such as 8 M urea are often used to unfold proteins during protein reduction and alkylation, direct protein digestion in this condition quickly deactivates commonly used enzymes. Moreover, normal online data-dependent MS/MS analyses adopt a single MS2 setting (often with unit mass resolution) for dissociation of several most abundant ions regardless of their charge states. Uniform setting is incompatible with electron-based dissociation of large peptides with a diverse charge state distribution. Compared to small peptides, large peptides are often highly charged and require tailored parameters for electron-based dissociation to achieve optimal fragmentation. In addition, large peptides require averaging high-resolution MS2 scans, which results in extended duty cycle, to compensate for decreased fragment signals due to more fragmentation channels.

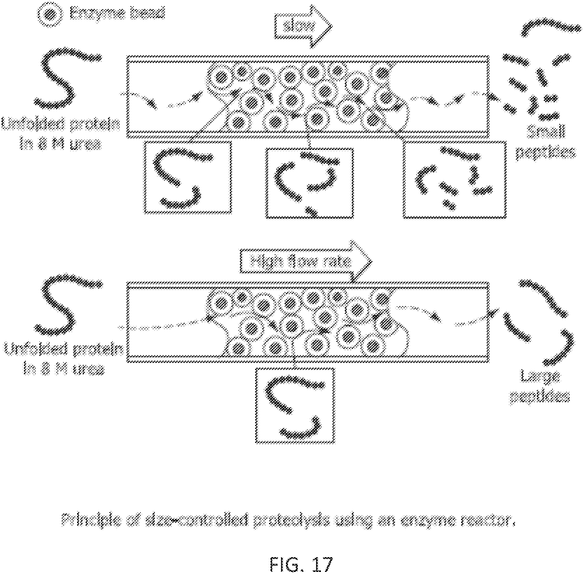

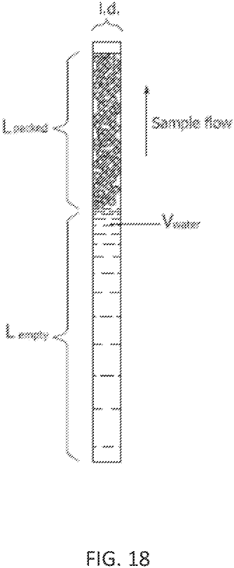

For example, in some embodiments, to hydrolyze a 150 kDa mAb into mainly 3-10 kDa peptide fragments for MS analysis, an enzyme reactor was prepared by packing a capillary column with 1 µm beads coated with aspergillopepsin I that had been covalently immobilized to the beads (see FIGS. 17 and 18, and Examples). Precise control of the sample flow rate as the sample passed through the column lead to determined residence time of the substrate protein in the reactor. A short residence time (t) results in a few cuts along the protein chain and ultimately the formation of large peptides. Pushing the protein through the enzyme reactor in <1 s breaks the protein into large peptides that facilitate mapping the sequence of apomyoglobin (17 kDa) and bovine serum albumin (66 kDa) by infusion electrospray ionization (ESI) MS.

Aspergillopepsin I, also known as protease type XIII, generally catalyzes the hydrolysis of substrate proteins in P1 and P1′ of hydrophobic residues, but also accepts Lys in P1. Rationale and advantages of using immobilized aspergillopepsin I for digestion in the presently disclosed enzyme reactors include, but are not limited to, the following:
1) aspergillopepsin I has sustained activity in 8 M urea at pH ~4. This extreme chaotropic condition can disrupt the higher-order structure of proteins to the most extent and allows for easy access of the protease to most regions of the substrate protein;
2) the broad protease specificity allows for near random chance of enzymatic cleavage along the unfolded protein chain; and
3) in-tube digestion by free aspergillopepsin I is difficult to quench due to the sustained activity of the protease in broad pH range. The enzyme reactor however automatically “quenches” proteolysis as the sample flows out of the column.

The features of immobilized aspergillopepsin I described above and in the Examples, along with the time-controlled digestion mode, resulted in the generation of mainly 3-10 kDa highly charged large peptides that facilitate online ETD MS/MS analysis. Also disclosed herein is alkylation of Cys residues with a reagent, N-(2-aminoethyl)maleimide (NAEM), prior to digestion. This alkylation reagent improves ETD characterization of peptides by adding additional basic groups to Cys. Selecting the most abundant about 40 large peptides for online MS/MS revealed near complete sequence of and multiple PTMs.

In some embodiments, digestion of a protein with a hydrolyzing agent or with a hydrolyzing agent in the presence of an inhibitor results in about 2 to about 20 fragments. In some embodiments, it generates about 5 to about 15 fragments. In some embodiments, it generates about 10 fragments. One of ordinary skill in the art will appreciate that the number of fragments refers to fragments with strong signals/high abundance, so the numbers referred might also be construed to be major fragments.

In some embodiments, the presently disclosed subject matter provides a liquid chromatography mass spectrometer system, method, and apparatus useful for rapid protein sequence analysis and detection of post-translational modifications. In some embodiments, the apparatus comprises an immobilized hydrolyzing agent. In some embodiments, the agent is immobilized to an aldehyde-functionalized particle. In some embodiments, the agent is a protease. In some embodiments, the system comprises an adjustable flow rate.

In some embodiments, the presently disclosed system is capable of analyzing a protein sample. In some embodiments, the system comprises an immobilized hydrolyzing agent, wherein the hydrolyzing reagent is selected from the group consisting of: aspergillopepsin I or a biologically active fragment or homolog thereof; a protease substantially identical to aspergillopepsin I or a biologically active fragment thereof of the protease; and a protease that is capable of hydrolyzing the protein sample under acidic and highly chaotropic conditions to generate peptides in the range of about 3 to about 10 kDa in mass. In some embodiments, the range is about 4 to about 9 kDa in mass. In some embodiments, the range is about 5 to about 8 kDa in mass. In some embodiments, the range is from about 6 to about 7 kDa in mass. In some embodiments, the hydrolyzing agent is aspergillopepsin I. In some embodiments, the hydrolyzing agent is immobilized on beads within a flow through column. In some embodiments, the highly chaotropic conditions consist of 8 M urea.

In some embodiments, the protein sample is an antibody sample. In some embodiments, the antibody sample is a therapeutic antibody sample. In some embodiments, the antibody sample is a bispecific antibody sample. In some embodiments, the protein sample comprises a protein of about 150 kDa in mass. In some embodiments, the pH is about 3.5 to about 4.0. In some embodiments, the concentration of the protein is about 1 µg/µL or less. In some embodiments, the concentration of the protein sample is about 0.2 µg/µL or less. In some embodiments, the concentration of the protein sample is less than about 0.1 µg/µL. In some embodiments, the concentration of the protein sample is between about 0.02 µg/µL and about 0.1 µg/µL. In some embodiments, the protein is disposed in the digestion buffer at a concentration of about 0.2 µg/µL or less or at a concentration of about 0.1 micrograms per microliter (µg/µL) or less. In some embodiments, the protein is disposed in said digestion buffer at a concentration of about 0.02 µg/µL to about 1 µg/µL, optionally about 0.05 µg/µL.

In some embodiments, the protein sample comprises an additive, wherein the additive is an inhibitor of the hydrolyzing agent (e.g., a competitive inhibitor of the hydrolyzing agent). In some embodiments, the concentration of the inhibitor is about the same as the concentration of the protein (i.e., the protein being characterized) in the protein sample. In some embodiments, the concentration of the additive is greater than the concentration of the protein. In some embodiments, the concentration of the additive is about 1.0, 1.5, 2.0, 2.5, or about 3 times the concentration of the protein. In some embodiments, the additive comprises a protamine. However, other inhibitors can be used to slow down the reaction rate, such as but not limited to, another protein or peptide, or a buffer. In some embodiments, the hydrolyzing agent inhibitor comprises at least one inhibitor selected from the group comprising guanidinium chloride, bovine serum albumin (BSA), and a protamine. The inhibitors have an effect on reaction rate. For example, the protamine inhibitor was shown to be useful in increasing the sequence coverage of bispecific antibodies, whose different segments have different concentrations under the conditions used for the reactor.

In some embodiments, urea is used as a chaotropic agent. In some embodiments, it is effective at a pH range of about 3.0 to about 5.0. In some embodiments, it is about 3.5 to about 4.0.

In some embodiments, protein denaturation is done in the absence of urea, and is done instead at high heat at temperatures up to about 100° C. In some embodiments, the digestion buffer comprises 0.5% acetic acid at temperatures up to about 100° C.

In some embodiments, a protease other than aspergillopepsin is used. In some embodiments, a protease that is active under weak basic conditions (e.g., pH 8-9) can be used. In some embodiments, an acid-cleavable surfactant, such as RapiGest, can be used to improve protein denaturation and digestion under weak basic conditions. Then, following digestion, acid can be added to the sample to degrade the surfactant so that the surfactant does not affect LC MS analysis. Proteases that work at pH 8-9 include Lys-C, Lys-N, Asp-N, and Glu-C. Additionally, if one of these proteases, such as Lys-N were immobilized into the column/reactor of the presently disclosed subject matter, high temperatures of about 70° C. can be used or buffers containing 50% acetonitrile can be used to improved protein denaturation.

In some embodiments, the presently disclosed subject matter comprises using time limited proteolysis (i.e., digestion) to produce 3 kDa -10 kDa fragments. In some embodiments, the presently disclosed subject matter comprises using a combination of ETD, CAD, and HCD tandem mass spectrometry to characterize the resulting peptides.

In some embodiments, the presently disclosed subject matter provides compositions and methods that disrupt the limitation in the art of conventional in-solution protein digestion that solely relies on enzyme specificity and extends the digestion condition to 8 M urea that favors unfolding of many compact proteins. In addition, the employment of aminoethylmaleimide as a Cys alkylating reagent enhances the charge states of peptides containing Cys and improved ETD MS². This strategy shows ability in digesting antibodies into 3-10 kDa peptides compared to in-solution digestion by LysC and AspN, and yields > 98% sequence coverage for an antibody. Moreover, PTMs on antibodies including pyroglutamic acid formation, oxidation, amidation, and glycation have been identified.

Other proteases can be used to practice the presently disclosed subject matter, such as in the context of micro-column enzyme reactors for generating large protein fragments (Switzar et al., Protein Digestion: An Overview of the Available Techniques and Recent Developments, J Proteome Res 2013; 12:1067-1077). Other useful proteases (specificities) include: Lys-N (n-terminal of Lys); Lys-C (c-terminal to Lys); and OmpT (between two consecutive basic residues Lys/Arg-Lys/Arg). Lys C works in 8 M urea (Choksawangkarn et al., Enrichment of Plasma Membrane Proteins Using Nanoparticle Pellicles: Comparison Between Silica and Higher Density Nanoparticles, JProteome Res 2013; 12:1134-1141). Lys-N works in both 8 M urea and 80% acetonitrile (Taoutas et al., Evaluation of Metalloenopeptidase Lys-N Protease Performance Under Different Sample Handling Conditions, J Proteome Res 2010; 9(8):4282-4288) and OmpT has been shown to cleave proteins into 6 kDa fragments (Wu et al., A Protease for ‘Middle-down’ Proteomics, Nat Methods 2012; 9(8):822-824).

It is disclosed herein that Lys-N digests proteins under harsh conditions that improved protein solubility or denaturation, including 8 M urea, 70° C., and buffer containing 50% acetonitrile.

In some embodiments, the presently disclosed subject matter discloses an apparatus to practice the methods of the presently disclosed subject matter. Disclosed herein are MS apparatuses and strategies which utilize an immobilized hydrolyzing agent, e.g., an aspergillopepsin I enzyme, an enzymatically active fragment thereof, or a polypeptide substantially identical to any of the foregoing, with broad specificity and consistent activity in highly chaotropic environment, (e.g. 6 M-10 M urea), to digest denatured proteins into large peptides via a size-control mode. Selecting a proper flow rate as the protein sample passes through the protease column precisely controls digestion time.

In some embodiments, the presently disclosed subject matter generates mainly medium size peptides of about 3 kDa to about 10 kDa. In some embodiments, the presently disclosed subject matter can be used to generate peptides of about 10 kDa to about 20 kDa. In some embodiments, the presently disclosed subject matter can be used to generate ultra-large size peptides of about 20 kDa to about 50 kDa. This includes 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49, and 50 kDa, and all numbers and fractions subsumed within that range.

In some embodiments, the presently disclosed subject matter generates mainly peptides in size ranges of about 1-20 kDa, 2-15 kDa, 3-12 kDa, 3-10 kDa, 3-9 kDa or 3-8 kDa, and includes all numbers and fractions subsumed within that range, from proteins. Peptides in this size range are favorable for protein sequencing when coupled with online LC-ETD MS/MS. In some embodiments, the employment of an alkylating agent (e.g., NAEM as a Cys alkylating reagent disclosed herein) improves ETD of peptides containing complementary determining regions (CDRs) by enhancing the peptide charge state.

In some embodiments, the presently disclosed subject matter can be used in conjunction with a multi-segment online LC-MS/MS strategy, to allow for the sequencing of a protein (e.g., a 150 kDa protein such as an antibody) with, for example, about 98% sequence coverage for an antibody. In some embodiments, the presently disclosed subject matter allows for the identification of multiple PTMs on proteins, such as antibodies, including, but not limited to, pyroglutamic acid formation, oxidation, amidation, deamidation, phosphorylation, methylation, acetylation, and glycosylation. One of ordinary skill in the art will appreciate that many PTMS can be identified and localized using the compositions and methods of the presently disclosed subject matter. In some embodiments, a PTM that is stable at about pH 3-4 is detected. Additionally, most PTMs that have been found in the art using LC MS should be applicable to the presently disclosed methods because the presently disclosed methods encompass a pH similar to that used for LC-MS.

In some embodiments, the presently disclosed subject matter involves an apparatus and method for rapid amino acid sequence analysis and characterization of large proteins such as antibodies or antibody-like molecules, membrane proteins, or large fragments of such proteins at the low picomole level. In some embodiments, the presently disclosed method involves: 1) reduction, alkylation and digestion of the protein sample while it is fully denatured in a solution that is highly chaotropic, e.g., 8 M in urea; 2) choosing a protease that functions under acidic conditions (e.g., a pH 3.9) and is that is not denatured in 8 M urea; (3) a flow-thru reactor constructed from 360 micrometer o.d. x 150 micrometer i.d. fused silica capillary equipped with a 2 mm Kasil frit and packed with aldehyde/sulfate latex beads (e.g., about 1 µm beads) covalently linked to the protease, aspergillopepsin I; (4) generation of peptide fragments in the 3-15 kDa mass range by control the sample flow rate through, and thus sample residency time (about 1-6 seconds) in, the capillary reactor; and (5) amino acid sequence analysis of the resulting fragments by nanoflow-HPLC interfaced to electrospray ionization on a tandem high resolution mass spectrometer equipped for both collision activated dissociation and electron transfer-dissociation. See also FIGS. 17 and 18.

In some embodiments, the presently disclosed subj ect matter provides for the calculation of flow rates and determination of digestion times. For example, one flow rate range of the presently disclosed subject matter is based on an 8-cm long enzyme reactor, and this range is proportional (has a linear relationship) to the length of the enzyme reactor. One of ordinary skill in the art will understand that the linear relationship can be used to make the calculations necessary when digesting a protein.

At least two factors can affect desired sample flow rate: IgG concentration (or other test protein) and the length of packed enzyme reactor. To produce peptides with a desired size range (i.e. medium size 3 kDa to 10 kDa) using a given enzyme reactor column, lower protein concentration employs a higher flow rate (i.e. less digestion time). For example, if 0.2 mg/mL (i.e. 1.35 pmol/microliter) IgG were being used, the factor that controls the general size of final peptides is the digestion time. In some embodiments, to achieve an optimal peptide size range of about 3 kDa to about 10 kDa, including all numbers and fractions subsumed within that range, one would need 5.7 sec digestion time, which is realized by flowing the sample at 4.5 microliter/min through an 8-cm long enzyme reactor. However, if an enzyme reactor longer than 8 cm is used, the sample will need to flow through the column faster in order to achieve a 5.7 s digestion time. Further, in some embodiments, additional considerations of the effects of concentration are brought to bear to provide improved performance, in accordance with the presently disclosed subject matter. The presently disclosed subject matter exemplifies additional concentration effects, in the data and figures and advantageously employs the concentration effects.

Additionally, in some embodiments, if a protein at a concentration of 0.2 mg/ml, such as alkylated IgG, were subjected to flow through a 8 cm long enzyme reactor, and in an effort to create IgG peptides with a size ranging from medium size (i.e. 3 kDa -10 kDa) to ultra-large size (i.e. 20 kDa-50 kDa), the digestion time would probably range from about 0.5 s to about 6 s. To realize this, the corresponding flow rate should be adjustable in the range of about 50 to about 4.0 µl/min. Flow rate can be calculated using Equation (3).

The presently disclosed subject matter does not require that a column be packed exactly at 8 cm every time. According to Equation (3), to achieve a certain digestion time, one can select the flow rate based on the actual length of packed enzyme reactor. This technique provides an advantage over the art. For example, if one would like to repeat the digestion with a certain digestion time (e.g. 5.7 s, 3 s, 1 s, etc.), one does not need to pack a new column with exactly the same enzyme reactor length as the previous column, which is not practical. Therefore, if the new column is half the length of the previous column, the flow rate should also drop to half in order to achieve the same digestion time.

### Principle of Size-controlled Proteolysis

Determination of the porosity (p) of the protease particles in an enzyme reactor. The porosity of the particles was determined by first loading the enzyme-column with water, then measuring the volume of the water trapped in the whole enzyme-column (including both the packed portion and the empty portion) using a 5-µL calibrated pipet as the water was pressure-pushed off the column. The porosity p was calculated according to Equation 2, where V_{water} is the volume of the water trapped in the whole column, V_{empty} is the volume of the portion with no protease particles packed, and V_{packed} is the volume of the portion of the column packed with protease particles (see Equations 1, 2, and 3 below). These equations can be adapted to other particle sizes, such as 1 µm, as described elsewhere herein.

$$t = \frac{π {\left(i . d . / 2\right)}^{2} {L}_{p a c k e d} p}{F}$$

$$p = \frac{{V}_{w a t e r} - {V}_{e m p t y}}{{V}_{p a c k e d}} \times 100 \%$$

$${V}_{e m p t y} = π {\left(\frac{i . d .}{2}\right)}^{2} {L}_{e m p t y}$$

$${V}_{p a c k e d} = π {\left(\frac{i . d .}{2}\right)}^{2} {L}_{p a c k e d}$$

$$t \left(s\right) = \frac{{L}_{p a c k e d} \left(c m\right)}{F \left(μ L / m i n\right)} \times 3.02$$

Principle of Time-controlled Protein Digestion. Pressure can be applied to drive the protein sample through the enzyme reactor packed with a certain length of protease particles (L_{packed}, which can be easily measured). Maintaining a stable pressure leads to constant flow rate (F) of the sample stream in the column, and consequently constant residence time for any moving cross-section of the flowing stream as it passes through the protease particles. Assuming there is no retention of proteins or peptides on the hydrophilic protease particles, the residence time (t) of any single protein “molecule” (here defined as a given protein molecule in either the starting intact form or its following hydrolyzed forms) should be the same as that of the stream cross section where the protein “molecule” exists. Based on this assumption, the residence time t, also defined as the on-column digestion time, for each single protein “molecule” can be calculated using Equation 1, where i.d. is the inner diameter of the capillary column, L_{packed} is the length of packed protease particles, F is the sample flow rate, and p is the porosity of the packed protease particles in the column (see Equation 2 for the determination of p). As p in the 150 µm i.d. column is a constant value (28.5% in this work) independent of other parameters, Equation 1 can be simplified to Equation 3. Thus, the digestion time t can be precisely controlled by maintaining a proper flow rate F that is proportional to L_{packed}. This is beneficial to repeating a time-controlled digestion using a new enzyme reactor with a different L_{packed} from before.

In some embodiments, the units for describing flow rate are µL/min. These units were used to determine the flow rate using a 5 or 10 µL calibrated pipette by collecting a certain volume of liquid flowing out of the column in 1 minute or half a minute and calculating the flow rate.

In some embodiments, the presently disclosed subject matter provides an apparatus and a method for sequencing proteins and detecting post-translational modifications. In some embodiments, a rapid method for sequence analysis of proteins of about 150 kDa is provided. In some embodiments, a protein of interest is denatured and then digested. In some embodiments, the protein is digested in urea. In some embodiments, the urea is used at a pH of about 4.0. In some embodiments, the concentration of urea is about 8 M. In some embodiments, the digestion is controlled by passing the protein sample in urea through a column comprising an immobilized protease using a precisely controlled digestion time. In some embodiments, the method generates fragments of about 3 kDa - 9 kDa.

The presently disclosed subject matter further provides steps of denaturing proteins.

In some embodiments, the flow rate is measured and adjusted by tuning the pressure applied to the column.

In some embodiments, the presently disclosed subject matter is useful for disulfide bond localization. In some embodiments, the protein digestion time is increased to enhance disulfide bond localization. In some embodiments, protein concentration is decreased to enhance disulfide bond localization.

In some embodiments, the alkylating agent is NAEM.

The amount of time for protein digestion can be varied to achieve different results as to disulfide bond localization. In some embodiments, longer digestion times are employed to localize disulfide bonds. In some embodiments, lower protein concentration is employed to localize disulfide bonds.

In some embodiments, a protein to be sequenced is denatured and then digested. In some embodiments, the protein is an antibody. In some embodiments, the antibody is a monoclonal antibody. In some embodiments, the antibody is a therapeutic antibody. In some embodiments, the antibody is a bispecific antibody. In some embodiments, the denatured proteins are reduced and alkylated. In some embodiments, the proteins are fully denatured.

In some embodiments, disulfides of a protein are reduced with tris(2-carboxyethyl)phosphine (TCEP). In some embodiments, the protein comprising reduced disulfides is alkylated. In some embodiments, the alkylating agent is NAEM. In some embodiments, the alkylated protein is diluted to about 0.2 µg/µL with urea. In some embodiments, the urea is used at about 8 M and a final pH of ~4. This solution is then used for on-column digestion of the protein. The protein is then subjected to size-controlled proteolysis by passing the sample through the column at a flow rate that is adjustable.

In some embodiments, the digestion buffer comprises urea. In some embodiments, urea is used at 8 M. In some embodiments, the pH of the buffer is about 4.

In some embodiments, the presently disclosed subject matter provides an enzyme reactor, also referred to as a chamber. In some embodiments, the enzyme reactor comprises a protease that has been immobilized (e.g., on particles or beads, such as 1 µm diameter polymeric particles or beads). Particle or bead size can range from about 1 µm to about 20 µm, including any size in between the endpoints of this range. In some embodiments, a column is prepared comprising immobilized protease. In some embodiments, the enzyme is a hydrolytic enzyme. In some embodiments, the enzyme is a protease. In some embodiments, the protease has broad specificity. In some embodiments, the protease with broad specificity is aspergillopepsin I.

In view of the structural/functional information available about aspergillopepsin I protein, one of skill in the art would be able to determine which fragments of the protein would be capable of being cleaved at hydrophobic residues in P1 and P1′, but also accepting Lys in P1 under highly chaotropic conditions. This is referred to herein as “aspergillopepsin I” activity.

In some embodiments, aspergillopepsin I is immobilized on aldehyde-functionalized particles by reductive amination under “salting out” conditions. In some embodiments, the aldehyde-functionalized particles are 20 µM particles. In some embodiments, the aldehyde-functionalized particles are smaller, e.g., about 1 µM diameter, particles. In some embodiments, the enzyme modified particles are suspended in water and packed into a fused silica capillary to form an enzyme reactor. In some embodiments, the fused silica capillary is 360 µM o.d. x 150 µM i.d. In some embodiments, the enzyme reactor can be from about 1 mm to about 15 cm long. In some embodiments, the reactor is from about 2 to about 14 cm long. In some embodiments, the reactor is about 8 cm long. One of ordinary skill in the art can readily determine the size of the reactor needed based on the methods disclosed herein.

The flow rate can be adjusted based on the time needed for digestion to occur. Factors to be considered include, for example, the length of the column or chamber or vessel being used, the inner diameter of the column, the length of the column, the volume of the column, the amount of hydrolyzing agent that is immobilized, the amount of protein to be passed through the column, the amount time that the protein should be in contact with the hydrolyzing agent, the particular hydrolyzing agent being used, the size of the protein or polypeptide being digested and the size of the peptides desired for analysis of the sequence, PTMs, or disulfide bond localization.

For disulfide bond location, a native mAb or another protein of interest can be subjected to the same procedure but with longer digestion times controlled by sample flow rate through the micro column reactor or lower protein concentration. Release of disulfide containing peptides from accessible regions of the folded protein occurs with short digestion times. The identity of two peptides connected by a disulfide bond can be determined using a combination of ETD and ion-ion proton transfer (IIPT) chemistry to read the two N-terminal and two C-terminal sequences of the connected peptides. (See: (i) Protein Identification Using Sequential Ion/Ion Reactions and Tandem Mass Spectrometry, Coon J J, Ueberheide B, Syka J E P, Dryhurst D D, Ausio J, Shabanowitz J, Hunt D F, Proc Natl Acad Sci USA, 2005 Jul. 5; 102(27):9463-8. PMCID: PMC1172258; (ii) Analysis of Intact Proteins on a Chromatographic Time Scale by Electron Transfer Dissociation Tandem Mass Spectrometry, Chi A, Bai D L, Geer L Y, Shabanowitz J, Hunt D F, Int. J. Mass Spectrom., 2007, 259, 197-203. PMCID: PMC1826913; (iii) Protein Derivatization and Sequential Ion-Ion Reactions to Enhance Sequence Coverage Produced by Electron Transfer Dissociation Mass Spectrometry, Anderson L C, English A M, Wang W-H, Bai D L, Shabanowitz J, and Hunt D F, Int J Mass Spectrom 2014, DOI: 10.1016/j.ijms.2014.06.023). In some embodiments, IIPT is not used. In some embodiments, the identity of two peptides connected by a disulfide bond can be determined using ETD without IIPT.

In some embodiments, more than one protease is used.

In some embodiments, the digestion occurs while a solution comprising the protein passes through the column.

In some embodiments, the digested peptides are less than about 10 kDa and greater than about 3 kDa or less than about 20 kDa and greater than about 10 kDa or less than about 50 kDa and greater than about 20 kDa.

In some embodiments, PTMs are selected from the group consisting of pyroglutamic acid formation, oxidation, amidation, and glycosylation.

In some embodiments, the protease is selected from the group consisting of a aspergillopepsin I, LysN protease (Lys-N), LysC endoproteinase (Lys-C), endoproteinase Asp-N (Asp-N), endoproteinase Glu-C (Glu-C) and outer membrane protein T (OmpT).

The presently disclosed subject matter provides advantages over current methods in the art. For example, the in-tube digestion method in the art mixes target proteins (e.g. IgG) with a protease in a ~1:20 mass/mass ratio. However, the in-tube digestion has drawbacks. Using the in-tube method it would be difficult to quench a digestion that utilizes aspergillopepsin I as the protease. This is because the digestion is active at pH 3-4, which is also the condition for the following LC-MS analysis after digestion. For in-tube digestion, one has to load the digest sample to the HPLC column while the digestion is still going on. If, for example, one were to perform a 20 min in-tube digestion for IgG, then one would need at least 5 min to load the digest onto the column, and this 5 min adds 25% error to the digestion time. Then, another 10 min is required for column washing. This can add another 50% error to digestion time. Adding together the total digestion time error could be 75%.

In contrast, the on-column digestion mode as disclosed herein quenches the digestion easily and the sample protein stops digestion immediately after it flows out of the enzyme reactor. This provides for accurate control of the flow rate (error within 5%) and for accurate control of the digestion time (error is also 5%), which directly leads to the size of the final peptides. In some embodiments, a syringe pump is used to push the protein sample through the column, which provides even more stable flow rate (flow rate error could be <1%).

The presently disclosed subject matter can include the use of both “time-controlled” and “size-controlled” digestion which is done rapidly in the enzyme reactor/chamber as described herein. However, the presently disclosed subject matter provides other advantages over the art as well, such as providing for analysis of lower concentration protein samples and by employing concentration effects and the use of hydrolyzing agent inhibitors to control protein digestion to provide desired fragments for characterization. In some embodiments, using the presently disclosed compositions, methods, system, and apparatus as described herein, the amount of time for the entire procedure for prepping a protein, digesting it, running it through the reactor, and having a sample ready for LC MS analysis is greatly reduced. Because the digestion time is so fast and the samples can be stored and are re-useable, the methods provide additional advantages. For example, a set up and run procedure to obtain digested protein can take only 45 minutes once the column/enzyme reactor is prepared. In some embodiments, once the column/enzyme reactor chamber is prepared, a test of sample flow rate is performed using a “blank” solution which contains only the buffer used to prepare the IgG sample. Then the bomb pressure can be adjusted to achieve the desired flow rate. This step takes 5-10 min. Then, the protein (e.g., IgG) sample is passed through the column using the same bomb pressure, and it flows with very similar (sometimes ~ 10% lower but still stable) flow rate as for the “blank” test. Typically, the first ~10 microliter of solution flowing-out is discarded as it contains buffer or diluted IgG digest from the dead volume of the column. Then up to 20 microliter digest sample is collected. The total time for processing the sample through the column and collecting the digested protein can be up to about 20 minutes. Also, there can be a need to collect 3 digest samples that correspond to 3 different digestion time (i.e. 3 different flow rates), which would yield peptides with medium, large, and ultra large sizes, and if that is done then the entire process from beginning to end can take only up to 45 minutes. Further advantages of this procedure over in-tube collection are provided below.

Contrary to the in-chamber (column) digestion procedure and apparatus disclosed herein, for in-tube digestion each digestion takes 10-30 min depending on the peptide size desired. To obtain three different IgG digest samples that yield peptides from medium size to ultra large size, it would take at least 1 hr because the 3 digestions cannot be done in parallel as you have to do one digestion and run the sample immediately, then later do another digestion, and so on. It should also be emphasized that each in-tube digestion allows only a single LC MS analysis because the digestion is continuous after an aliquot of the digest sample is loaded to the HPLC for LC MS analysis. The rest of the IgG digest can be discarded after sample loading. In contrast, the presently disclosed method creates digest samples that can be stored and are reusable (up to 20 times LC MS analyses for a 20 µL digest sample). Considering all the above factors, the following estimation is provided:

Therefore, for a given new protein sample (such as IgG), in some embodiments, it is desirable to create three digest samples that correspond to three peptide size classes: medium size (3-10 kDa), large size (10-20 kDa) and ultra-large size (20-50 kDa). With the on-column digestion system disclosed herein, only 45 min is needed for preparation of the sample and its digestion, and the samples obtained after passing through the column provide enough material for up to 20 times LC-MS analysis/sample. However, using the in-tube digestion known in the art, the procedure can allow up to 5 LC MS analyses/sample, and this will involve three separate procedures (at least 1 hr. x 3 x 5=15 hrs total time for in-tube digestions) for a total of 15 hours. Based on this comparison and the results described herein, the present method is referred to as “rapid” relative to other methods and apparatuses used in the art for the characterization of proteins.

Although some proteins can be denatured for the most desirable result using the methods of the presently disclosed subject matter, the presently disclosed subject matter also encompasses the use of proteins that are not denatured before being dissolved in a digestion buffer of the presently disclosed subject matter. For example, one of the purposes of denaturing a protein as disclosed herein is to cause the molecule to be as linear as possible, so that the chances of digesting different regions of the protein are equal from one site to another. However, if the protein is natively very flexible (such as proteins that do not crystallize, like casein), denaturation using urea would not be needed and the protein can then be subjected to flow through the reactor by dissolving it in a simple buffer such as an acid buffer.

In some embodiments, the presently disclosed subject matter provides compositions and methods for characterizing the native structure of a protein such as IgG (e.g. localization of the disulfide bonds in IgG), or other highly folded proteins, by preserving the structure of the protein in its native state. As demonstrated herein, intact unalkylated antibody (e.g., a bsAb) in 8 M urea can be digested to generate ultra-large peptides that contain disulfide bond. Using a non-denatured condition can sometimes be useful for this type of study. However, without denaturation, the digestion will occur preferably to the most flexible region of a protein and should result in a simple final digest.

The presently disclosed subject matter further provides compositions and methods useful for preparing a reaction chamber.

The presently disclosed subject matter further provides a kit for practicing the methods disclosed herein. The kit can comprise reagents as disclosed herein, compositions as disclosed herein, and an apparatus as disclosed herein. The kit can also comprise components needed to build all or part of the apparatus. The kit can comprise instructional material for practicing the methods, building and/or using the apparatus, and instructions for use of the system of the presently disclosed subject matter.

Various techniques and methods for the use of mass spectrometry, etc. are known in the art and can be found in, for example, U.S. Pat. Application Publication No. 2021/0156792 (Syka et al.), U.S. Pat. No. 8,692,187 (Hunt et al.), U.S. Pat. No. 7,749,769 (Hunt et al.), U.S. Pat. No. 7,534,622 (Hunt et al.), and U.S. Pat. No. 8,119,984 (Shabanowitz et al.).

Other embodiments of the presently disclosed subject matter will be apparent to those skilled in the art based on the disclosure and embodiments described herein. It is intended that the specification and examples be considered as exemplary only, with a true scope and spirit of the presently disclosed subject matter being indicated by the following claims.

The presently disclosed subject matter is now described with reference to the following Examples. Without further description, it is believed that one of ordinary skill in the art can, using the preceding description and the following illustrative examples, make and utilize the presently disclosed subject matter and practice the claimed methods. The following working examples therefore, are provided for the purpose of illustration only and specifically point out the preferred embodiments of the presently disclosed subject matter, and are not to be construed as limiting in any way the remainder of the disclosure. Therefore, the examples should be construed to encompass any and all variations which become evident as a result of the teaching provided herein.

### EXAMPLES

The following Examples provide illustrative embodiments. In light of the present disclosure and the general level of skill in the art, those of skill will appreciate that the following Examples are intended to be exemplary only and that numerous changes, modifications, and alterations can be employed without departing from the scope of the presently disclosed subject matter.

### Example 1

### Methods and Materials

Materials. Equine apomyoglobin from skeletal muscle, lysozyme chloride from chicken egg white, β-lactoglobulin from bovine milk, Aspergillopepsin I, N-(2-aminoethyl)maleimide trifluoroacetate salt (NAEM), tris(2-carboxyethyl)phosphine hydrochloride (TCEP), and sodium cyanoborohydride were purchased from Sigma-Aldrich (St. Louis, Missouri, United States of America). Protamine sulfate salt from salmon was provided as a gift, but can also be purchased from Sigma-Aldrich (St. Louis, Missouri, United States of America). 1 µm aldehyde/sulfate latex beads were purchased from Invitrogen (Carlsbad, California, United States of America). The IgG-like bispecific antibody biosimilar of ACE-910 (emicizumab) was purchased from Creative Biolabs (Shirley, New York, United States of America).^{19,20} The cysteine protease IdeS (sold under the tradename FABRICATOR™) was purchased from Genovis (Genovis Inc., Cambridge, Massachusetts, United States of America). All other materials were purchased from Sigma-Aldrich (St. Louis, Missouri, United States of America) unless otherwise noted.

Enzyme Reactor Assembly. Conjugation of Aspergillopepsin I to 1 µm aldehyde/sulfate solid sphere beads was performed as previously described.¹³ Briefly, ~10 µg of beads were conjugated with Aspergillopepsin I through the reaction of the aldehyde groups on the beads with any free amine present on Aspergillopepsin I to form a Schiff base. The Schiff base was selectively reduced with sodium cyanoborohydride. Free aldehyde groups on the beads were blocked using Tris and reduced in the same manner. The beads were washed with water between each step and were stored dry at 4° C.

Sample Preparation. One nmol aliquots of equine apomyoglobin samples were reconstituted to the desired concentration using 8 M urea in 50 mM ammonium acetate buffer at pH 4 (digestion buffer). Stock solutions of chicken lysozyme, bovine β-lactoglobulin, and bovine trypsinogen were prepared at ~5 µg/µL using 0.1% acetic acid in water. Individual protein samples were prepared by evaporating an aliquot to dryness. Samples were reduced by reconstituting in TCEP in 8 M urea and 0.5% acetic acid in water and alkylated with NAEM in 8 M urea and 0.5 M ammonium acetate in water for alkylation.¹³ The reduced and alkylated samples were then brought to the desired concentration using digestion buffer.

A stock solution of protamine sulfate from salmon was prepared at ~10 µg/µL in water. When used to treat a sample, the final concentration of protamine was 0.2 µg/µL.

bsAb Preparation. ~8.5 µg of bispecific antibody was first digested with IdeS to cleave the antibody below the hinge region.²¹ The digestion of the bsAb with IdeS was performed so that subunits for digestion were similar in terms of molecular weight. The results of the digestion were evaporated to dryness. Reduction and alkylation of the bsAb with TCEP and NAEM was carried out as described above. See FIG. 1B. The final volume was diluted to 0.2 µg/µL with 42 µL digestion buffer. This procedure was repeated for a protamine treated digestion with the exception that the final volume was brought to 41 µL and 0.8 µL of 10 µg/µL protamine was added into the sample for a final bsAb concentration of ~0.2 µg/µL and protamine concentration of ~0.2 µg/µL.

Enzyme Reactor Digestion. A 2 mm packed bed of Aspergillopepsin-conjugated beads was prepared in a fritted 360 µm O.D. x 150 µm I.D. fused silica capillary. Each protein sample at each concentration was digested at various flow rates for a range of calculated digestion times as described previously (see Table 1, below, for exact measurements).¹³ Following digestion, samples were diluted with digestion buffer to ~1 pmol/µL (0.016 µg/µL) if necessary.

Chromatography and Mass Spectrometry. An Agilent Technologies (Palo Alto, California, United States of America) 1100 Series binary HPLC system coupled to an in-house modified Thermo Scientific LTQ-FT Ultra mass spectrometer (San Jose, California, United States of America) was used for evaluation of all standard protein digests.²² Digestion products were pressure-loaded onto an analytical column packed with 10 cm of 3 µm diameter, 1000 Å PLRP-s packing material (Polymer Laboratories, Church Stretton, United Kingdom) within a 360 µm O.D. x 75 µm I.D. fused silica capillary.^{23,24} Samples were washed with 0.3% formic acid in water (Solvent A) for 1 hour for desalting. Peptides were gradient eluted with a gradient of 0-25-55-100% Solvent B in 0-5-35-40 minutes. (Solvent B = 72% ACN, 18% IPA, 10% water, 0.3% formic acid.) Following a 100,000 resolution MS¹ scan, the top 3 peptides were dissociated by collisionally activated dissociation (CAD) and 45 ms of electron transfer dissociation (ETD). All MS² scans were analyzed in the ion trap.

For bispecific antibody digestions, the HPLC system was coupled to a mass spectrometer sold under the tradename ORBITRAP FUSION™ TRIBRID™ (Thermo Finnigan, LLC, San Jose, California, United States of America). Approximately 400 nL (~400 fmol) of the digestions were pressure-loaded onto the same PLRP-s column. The column was rinsed for ~45 min with 100% A to remove salts from the column. Peptides were eluted with a gradient of 0-25-50-100%B in 0-5-80-85 minutes at 50° C. A tiered data dependent method was employed as described previously to detect and fragment the eluting peptides. ¹³ In short, all the ions in the MS¹ scan were sorted into three priorities for subsequent fragmentation while tailoring the dissociation method to best suit the charge density of the precursors (high-capacity ETD for high charge density species; collisional dissociation for low charge density species).²⁵ Large ions were first priority and were fragmented with ETD (10-24 charges; 500-925 m/z; calibrated ETD reaction time) and higher-energy collisional dissociation (HCD) (8-24 charges; 1100-1500 m/z; 25±3% normalized collision energy (NCE)) for a high resolution (120 K) MS² scan with one additional microscan. Medium ions were second priority and were fragmented by ETD (5-9 charges; 300-925 m/z; calibrated ETD reaction time) and HCD (4-7 charges; no m/z restraints; 25±3% NCE) for a high resolution (60 K) MS². Small ions were the lowest priority and were fragmented by ETD (3-4 charges; no m/z restraints; calibrated ETD reaction time) and CAD (2-3 charges; no m/z restraints; 30% NCE) in the ion trap for low resolution MS² scans.

Data Analysis. For standard protein digestion results, data files were searched against the known sequence by individual dissociation type following conversion to .MGF files using software sold under the tradename BYONIC™, version 3.3.3 (Protein Metrics, Cupertino, California, United States of America). Manual verification of peptides was performed on a subset of peptides from each analysis. Spectra were averaged using Thermo Qual Browser version 4.0.27.10. (Thermo Fisher Scientific, Waltham, Massachusetts, United States of America).

For bispecific antibody digestion results, data files were searched using software sold under the tradename BYONIC™ Bwithin Proteome Discoverer (version 2.2.0.386) (Protein Metrics, Cupertino, California, United States of America) as described previously.¹³ Manual verification of peptides was performed to confirm the fragment ions observed. ProSight Lite (version 1.4) was used to produce the fragment ion map figures in this paper to display the fragment ions observed during manual verification. Calculation of all peptide abundances was performed using Proteome Discoverer. Peptides identified in the BYONIC™ (Protein Metrics, Cupertino, California, United States of America) searches following a 1% FDR cut off were grouped using a 10 ppm tolerance, a minimum S/N ratio of 5, and quantified by mass area. Only peptides with a confidence value of High from Proteome Discoverer were used. Pairwise ratios were calculated of the treated over untreated samples with a maximum ratio value of 500.

Table 1: Exact measurements used for the determination of digestion times. Times were calculated using the equation:

$$\begin{matrix}\frac{b e d l e n g t h \left(c m\right)}{f l o w r a t e \left(\frac{μ \text{} L}{m i n}\right)} \times π \times {\left(\frac{i . d . \left(c m\right)}{2}\right)}^{2} \times p \times 60000 \left(\frac{s e c \times μ \text{} L}{m i n \times m L}\right) \\ = D i g e s t i o n T i m e \left(s\right)\end{matrix}$$

where i.d. is the inner diameter of the fused silica, p is the porosity of the packed bed (28.5% for 1 µm beads), and 60000 is a conversion factor. All protamine concentrations are at 0.2 µg/µL.

| Protein | Concentration (µg/µL) | Bed Length (cm) | Flow Rate (µL/min) | Digestion Time (s) |
|---|---|---|---|---|
| Apomyoglobin | 0.2 | 0.20 | 0.60 | 1.01 |
| Apomyoglobin | 0.1 | 0.15 | 0.47 | 0.96 |
| | | | 0.57 | 0.79 |
| Apomyoglobin | 0.05 | 0.15 | 0.46 | 0.98 |
| | | 0.11 | 0.90 | 0.37 |
| Apomyoglobin | 0.02 | 0.20 | 0.62 | 0.97 |
| Apomyoglobin + Protamine | 0.2 | 0.19 | 0.60 | 0.96 |
| Apomyoglobin + Protamine | 0.1 | 0.19 | 0.62 | 0.93 |
| Apomyoglobin + Protamine | 0.05 | 0.21 | 0.64 | 0.99 |
| Apomyoglobin + Protamine | 0.02 | 0.19 | 0.62 | 0.92 |
| Chicken Lysozyme | 0.2 | 0.20 | 0.61 | 0.99 |
| Chicken Lysozyme | 0.05 | 0.20 | 0.61 | 0.99 |
| Chicken Lysozyme | 0.02 | 0.20 | 0.58 | 1.04 |
| Chicken Lysozyme + Protamine | 0.05 | 0.19 | 0.55 | 1.04 |
| Chicken Lysozyme + Protamine | 0.02 | 0.20 | 0.57 | 1.06 |

TABLE 1 (contd.)

| Protein | Concentration (µg/µL) | Bed Length (cm) | Flow Rate (µL/min) | Digestion Time (s) |
|---|---|---|---|---|
| β-lactoglobulin | 0.2 | 0.20 | 0.78 | 0.98 |
| β-lactoglobulin | 0.05 | 0.19 | 0.62 | 0.92 |
| β-lactoglobulin | 0.02 | 0.20 | 0.62 | 0.97 |
| β-lactoglobulin + Protamine | 0.05 | 0.20 | 0.62 | 0.97 |
| β-lactoglobulin + Protamine | 0.02 | 0.20 | 0.66 | 0.92 |
| Protamine | 0.2 | 0.19 | 0.64 | 0.9 |
| Trypsinogen | 0.2 | 0.20 | 0.61 | 0.99 |
| Trypsinogen | 0.05 | 0.21 | 0.63 | 1.01 |
| Trypsinogen | 0.02 | 0.20 | 0.62 | 0.97 |
| Trypsinogen + Protamine | 0.05 | 0.20 | 0.62 | 0.97 |
| bsAb | 0.2 | 0.21 | 0.66 | 0.96 |
| bsAb + Protamine | 0.2 | 0.19 | 0.54 | 1.06 |

TABLE 2

Sequences of Salmine.

| M+H (Da) | Sequence | SEQ ID NO: |
|---|---|---|
| 4234.59 | PRRRRRSSSR PIRRRRRPRA SRRRRRGGRR RR | 9 |
| 4317.68 | PRRRRSSRRP VRRRRRPRVS RRRRRRGGRR RR | 10 |
| 4248.61 | PRRRRSSSRP VRRRRRPRVS RRRRRRGGRR RR | 11 |
| 4062.54 | PRRRRASRRI RRRRRPRVSR RRRRGGRRRR | 12 |

### Example 2

### Results and Discussion

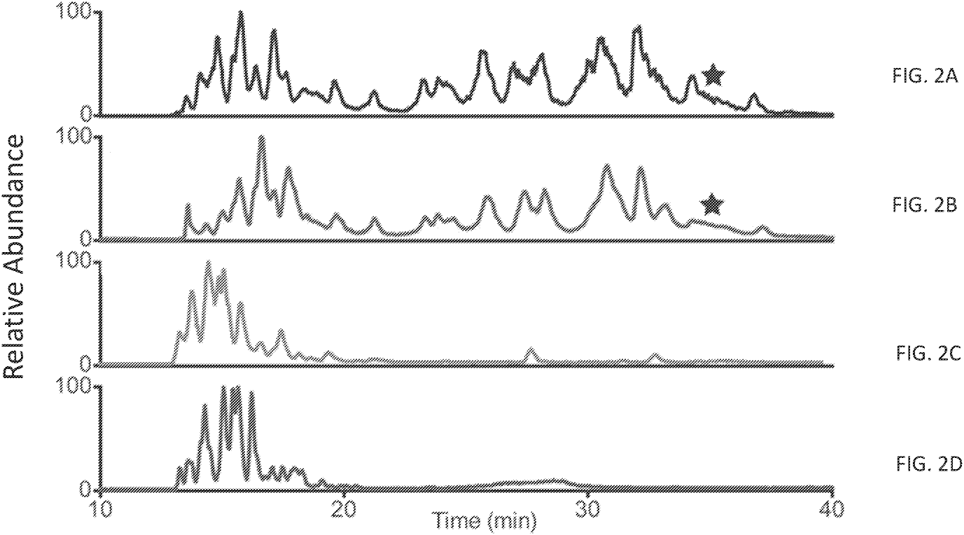

The Number of Enzymatic Cleavages Increases as Concentration Decreases. The effects of concentration on the resulting digestion using the Aspergillopepsin I reactor were initially explored using apomyoglobin as a standard. Previously, the reactor has been used to digest proteins at a concentration of 0.2 µg/µL (~11.8 pmol/µL).^{12,13} Concentrations of 0.1 µg/µL (~5.9 pmol/µL), 0.05 µg/µL (~2.9 pmol/µL), and 0.02 µg/µL (~1.2 pmol/µL) apomyoglobin were digested using the enzyme reactor at various digestion times to evaluate how the resulting peptide mixture changed. When the concentration of protein in the sample was decreased, the abundance of larger peptides decreased and the abundance of smaller peptides increased. See FIGS. 2A-2D. Without being bound to any one theory, this implies that as the protein analyte concentration decreases, the number of cleavages by Aspergillopepin I increases. In other words, at concentrations below 0.1 µg/µL, the reactor is diffusion limited.

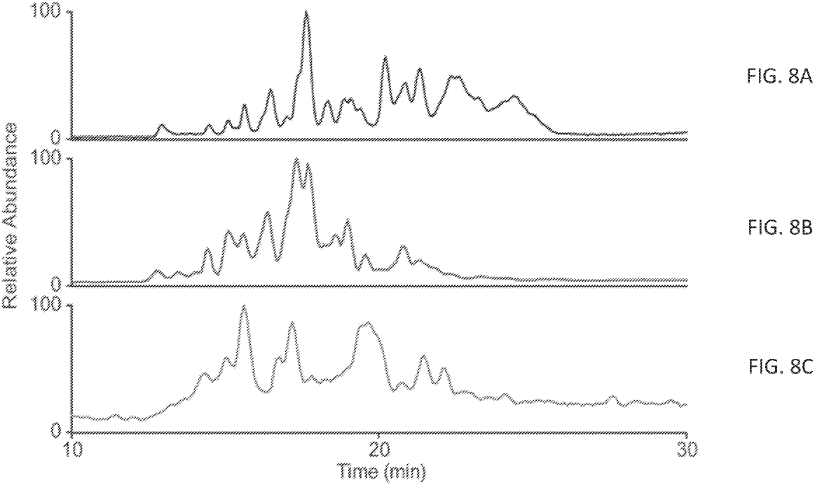

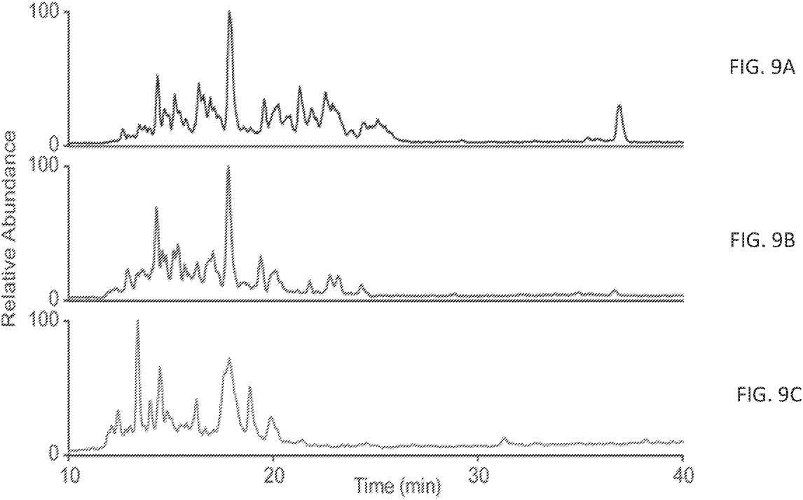

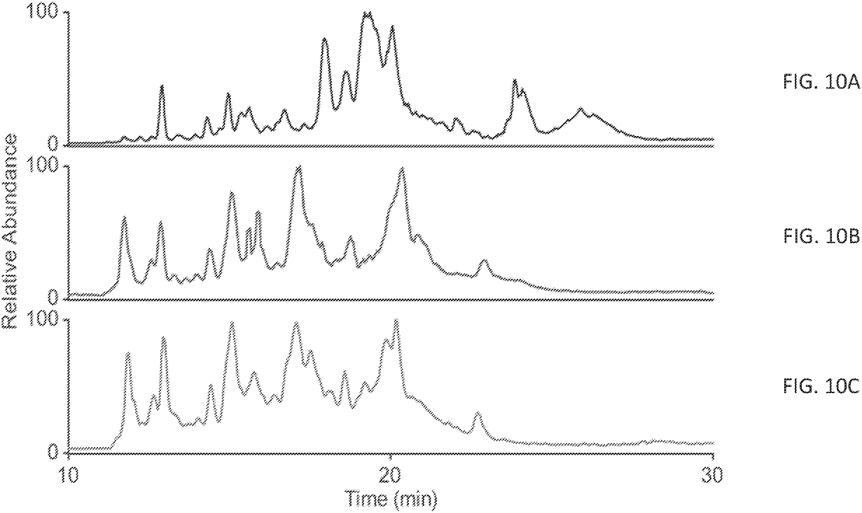

Enzyme reactors that are diffusion limited are dependent on the ability of the substrate to diffuse to the enzyme and for the products to diffuse away from the enzyme.²⁶⁻³⁰ Since Aspergillopepsin I is nonspecific, all products of an enzymatic cleavage are also substrates for additional cleavages. This can account for the increase in abundance of smaller peptides. Immobilized reactors can also be catalytic limited, in which all active sites of the enzyme are occupied by a substrate.³⁰ When all the active sites are occupied, products have time to diffuse away from the enzyme’s active site and therefore do not react repeatedly. At concentrations from 0.1 - 0.2 µg/µL, the reaction is closer to being catalytic limited as evidenced by the preservation of large peptides. These concentration effects were reproduced with other proteins, such as chicken lysozyme, bovine β-lactoglobulin, and bovine trypsinogen. See FIGS. 8A-8C, 9A-9C, and 10A-10C.

The chromatograms appear to indicate that apomyoglobin is most affected by the change in starting concentration. Many factors likely affect the digestion with the Aspergillopepsin I immobilized reactor. The structures of apomyoglobin (Protein Data Bank (PDB) entry: 1YMB⁴⁰), chicken lysozyme (PDB: 1DPX⁴¹), β-lactoglobulin (PDB: 1BEB⁴²), and bovine trypsinogen (PDB: 1TGB⁴³) can contribute to how sensitive the proteins are to these concentration effects. Apomyoglobin consists primarily of α-helices with no β-sheets, whereas Chicken Lysozyme, β-lactoglobulin, and Bovine Trypsinogen all contain β-sheets to some extent. Without being bound to any one theory, it is believed that α-helices are more effected by the starting concentration than β-sheets. The accessibility of the enzyme to the cleavage site can be impacted by the secondary structure of the protein of interest, causing these structural effects.

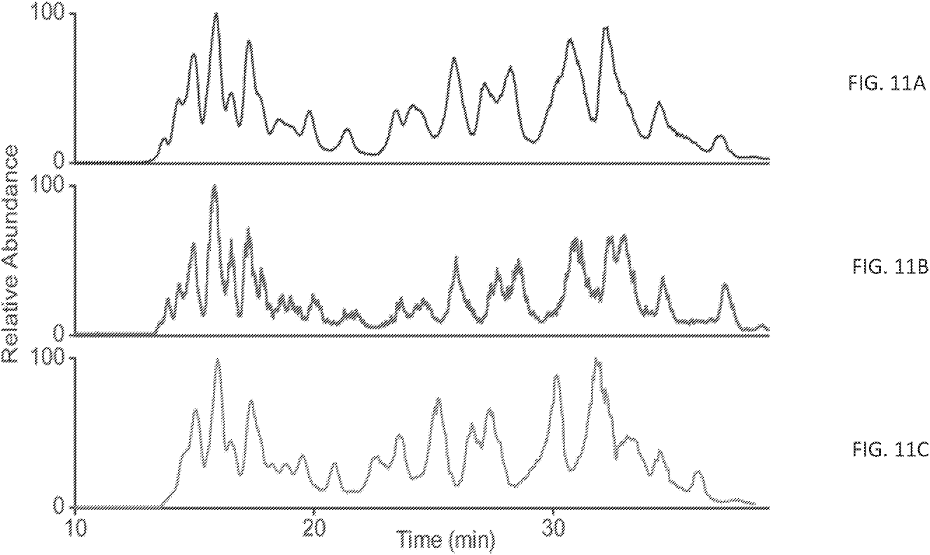

Although it can be useful to use the enzyme reactor with a sample concentration at 0.2 µg/µL, not all biological samples can be concentrated thusly. Therefore, methods of mitigating these concentration effects were explored to make the enzyme reactor amenable to a variety of sample concentrations. One way to compensate for the diffusion limited state is to decrease the amount of time the sample is in contact with the reactor, i.e. increase the flow rate. It was found that digestion times of about 0.7 s and about 0.4 s for the 0.1 µg/µL and 0.05 µg/µL samples, respectively, were comparable to that of an about 1 s digest at 0.2 µg/µL. See FIGS. 11A-11C. Based on this trend, a comparable digestion for a 0.02 µg/µL sample would be about 0.2 s. To obtain such short reaction times, higher flow rates or lower concentrations of enzyme in the form of a shorter bed length can be employed. However, as a packed bed as small as 2 mm was already being used, it was considered potentially difficult to pack a shorter bed with high reproducibility due to systematic errors in measurements. Furthermore, high flow rates can use an increase in the applied pressure through the enzyme reactor which can be incompatible with the current system construction (e.g. the frit cannot withstand pressures > about 750 psi). Consequently, other methods of compensating for the diffusion limited reaction were evaluated, such as using a competitive inhibitor. A desirable competitive inhibitor would be a peptide/protein with many hydrophobic or lysine residues.

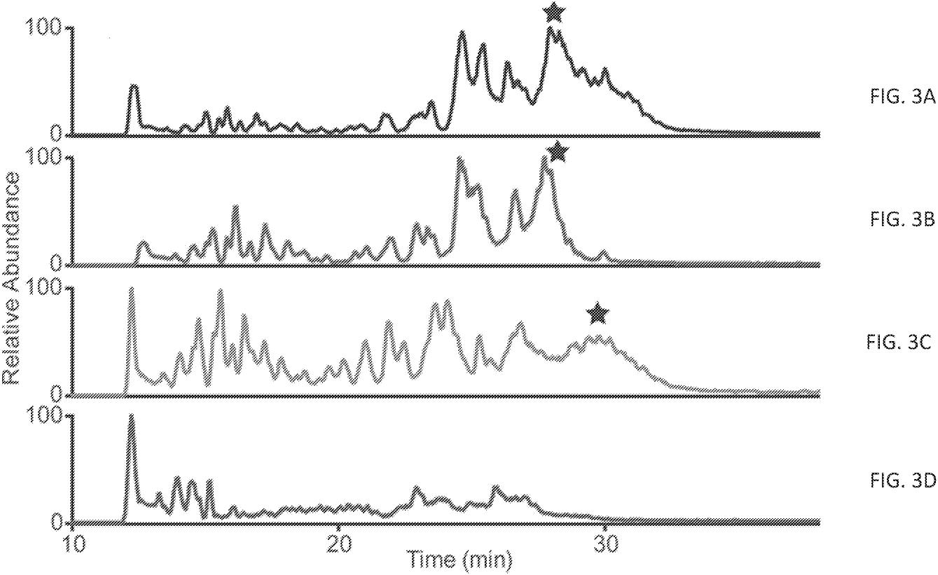

Protamine from Salmon Competitively Inhibit the Rate of Digestion. Previous experiments showed that Aspergillopepsin I favors arginines in the P1 position in addition to lysines.^{12,13} Therefore, if a protein with a high number of basic residues is added to a sample, the enzyme should preferentially cleave the basic protein over the sample protein. A mixture of protamine from salmon (salmine) was used to test this hypothesis. Protamine is a member of sperm nuclear basic proteins that replace histones towards the end of spermatogenesis.³¹⁻³³ These proteins are typically less than 100 amino acids in length with arginine representing a vast majority of the residues. The four major protamines in salmine are 30-32 amino acids in length with ~70% of those residues being arginine (see Table 2, below, for the amino acid sequences of salmine).³⁴ This mixture of protamines was added to variously concentrated apomyoglobin samples such that the final concentration of protamine was 0.2 ug/uL (47.4 pmol/uL). The chromatograms in FIGS. 3A-3D were produced by digesting apomyoglobin samples treated with protamine for about 1 s.

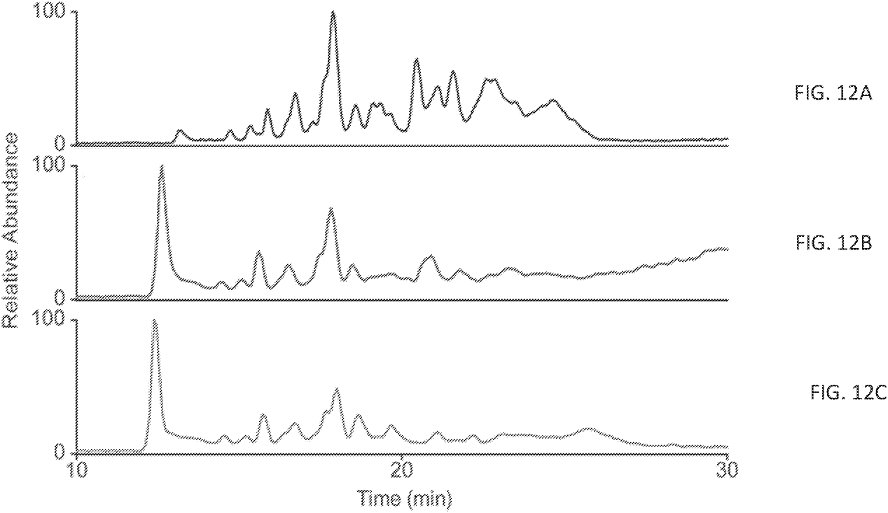

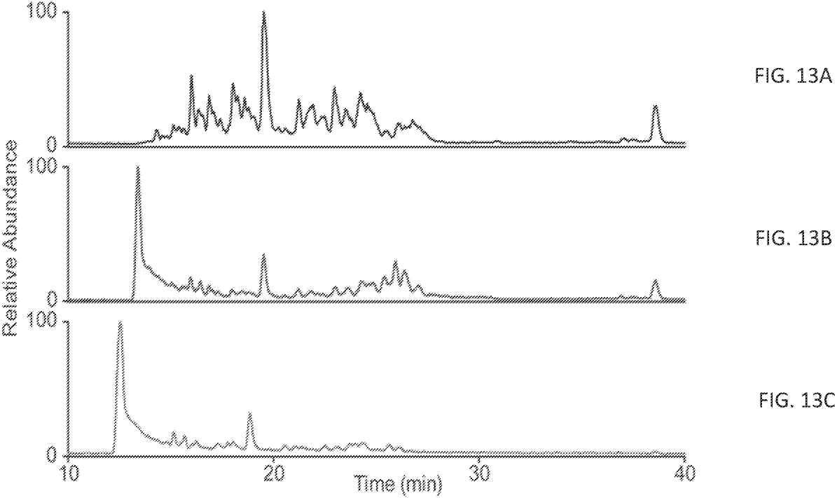

A comparison of the chromatograms of apomyoglobin digestions with (see FIGS. 3A-3D) and without (see FIGS. 2A-2D) protamine treatment shows that the abundance of high molecular weight products increased after treatment with protamine, including the abundance of undigested apomyoglobin. Treating samples where the low concentrations caused a diffusion limited enzymatic digestion with protamine (see FIGS. 3C and 3D) resulted in a digestion profile that looks very similar to that of the 0.2 µg/µL without protamine. See FIG. 2A. Therefore, the introduction of protamine to the diffusion limited system resulted in digestion profiles similar to the profiles generated by more highly concentrated digestions. When protamine was added to proteins at concentrations that were closer to catalytic limited digestion (see FIGS. 3A and 3B), the resulting chromatogram gave a high abundance of undigested apomyoglobin. This implies that protamine is slowing down the rate of digestion of apomyoglobin regardless of concentration. This phenomenon was reproduced for 0.05 µg/µL digestions of chicken lysozyme and β-lactoglobulin. See FIGS. 12A-12C and 13A-13C.

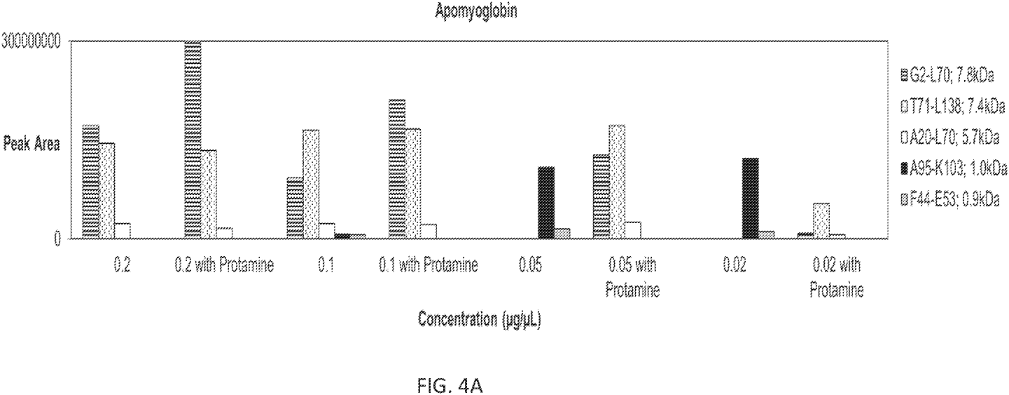

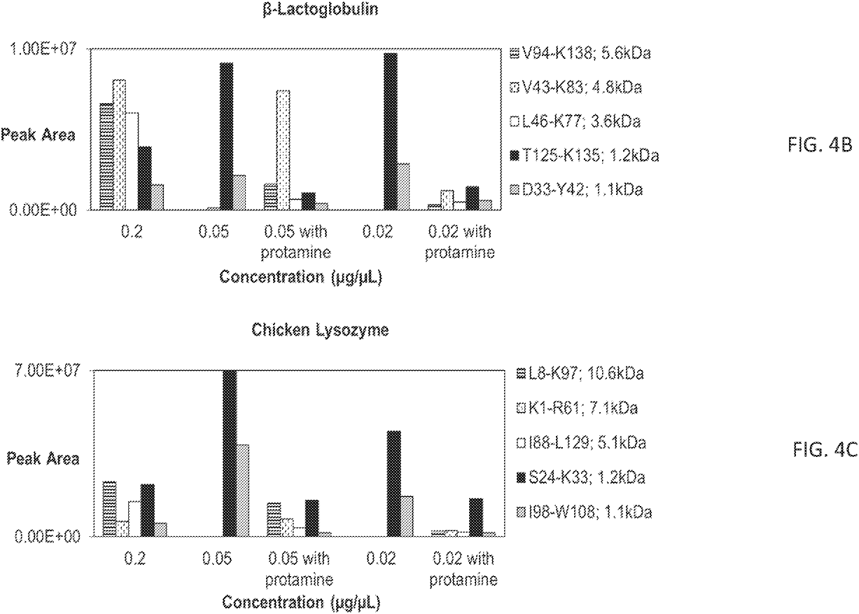

FIGS. 4A-4C depict this trend by tracking the abundance of a selection of peptides across the concentrations tested for all proteins. Adding protamine provides the Aspergillopepsin I enzyme reactor to be used on samples an order of magnitude lower in concentration in comparison to previous work.^{12,13} Ultimately, this provides a greater range of samples with a known concentration to be digested.

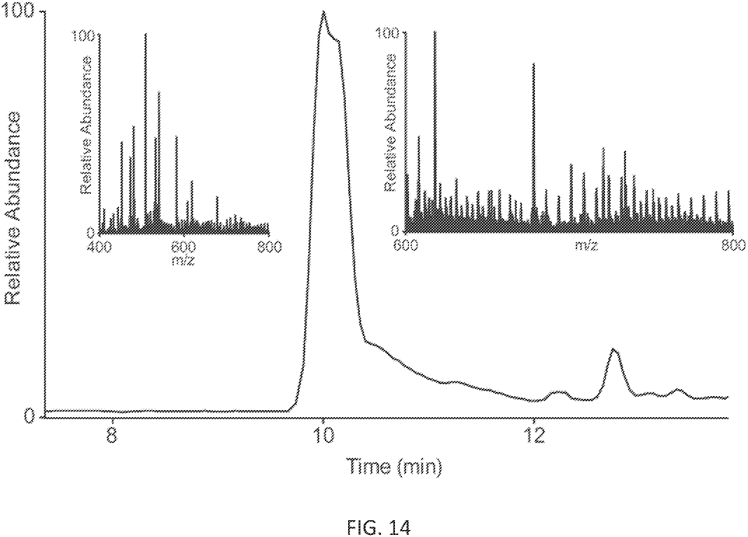

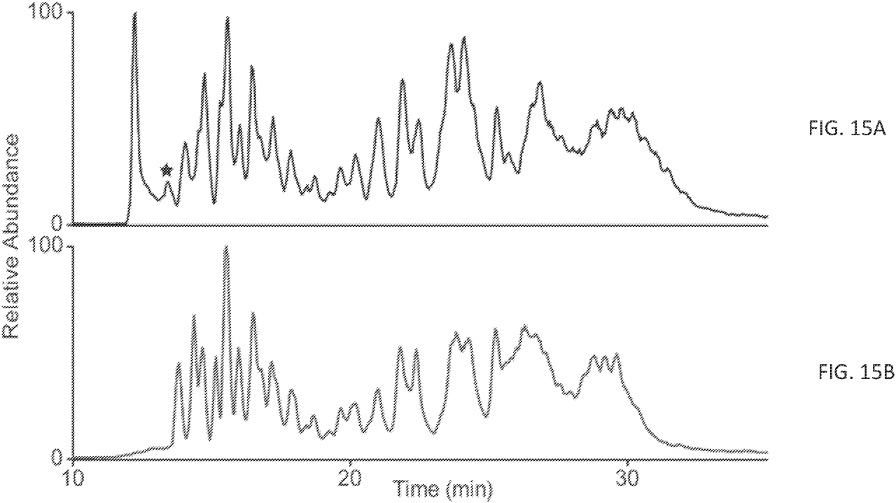

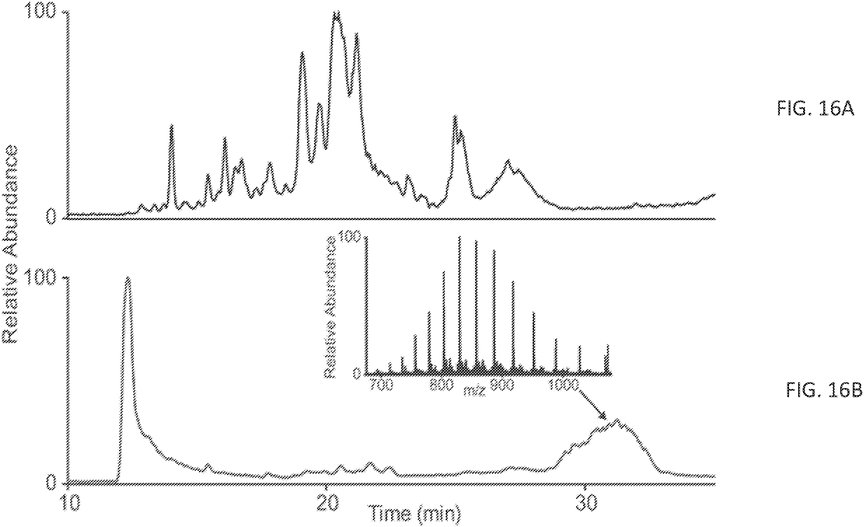

Protamine was digested by itself at 0.2 µg/µL to evaluate the digestion without additional proteins present. See FIG. 14. Following an approximately 1 s digestion, a significant abundance of undigested protamine was present in addition to multiple digestion products at lower abundance. This digestion pattern was also observed when standard proteins were introduced to the sample. Therefore, it is believed that protamine was competing with the analyte for access to Aspergillopepsin’s active site. Since diffusion rate generally decreases as molecular weight increases, protamine is capable of diffusing to and from the active site faster than the analyte protein.³⁵⁻³⁷ Protamine’s high diffusion rate in combination with the high abundance of preferential cleavage sites can allow it to take up active sites to prevent the protein of interest from being continually digested.

Protamine and its respective digestion products were the first species to elute from the column during analysis due to their high hydrophilicity. Additionally, it is possible that some digestion products from protamine are not retained on a reversed-phase column. These protamine species can be removed during the desalting step prior to analysis. The vast majority of these species were washed off the column by using a small percentage of organic solvent, in this case 4% solvent B. See FIGS. 15A and 15B. However, peptides from the protein of interest that elute with the small percentage of organic can also elute during this rinse step. Peptides that elute in this region are typically low in molecular weight and, as a result of the nonspecific digestion, are usually also found within larger peptides. Since the addition of protamine to the sample increases the abundance of these larger peptides, the loss of the small peptides during the rinse with organic does not yield a loss of sequence coverage.

As shown in FIGS. 4A-4C, treating samples with protamine for diffusion limited digestions reduced the rate of digestion for the majority of proteins. However, the amount the digestion rate was reduced varied between proteins. When bovine trypsinogen was digested at 0.05 µg/µL (about 2.1 pmol/µL) with protamine, the resulting chromatogram showed the digestion products of protamine, a high abundance of undigested trypsinogen, and very low abundance trypsinogen digestion products. See FIGS. 16A and 16B. Since the protein was reduced, alkylated, and in acidic pH and 8 M urea, trypsinogen should not be in its active form. Therefore, without being bound to any one theory, the addition of protamine could be interacting with trypsinogen to prevent it from being digested by the enzyme. This lack of digestion was not reproduced on the array of proteins tested; however, there is a possibility that other proteins can produce the same result as trypsinogen.

Unambiguous Sequence Coverage of a Bispecific Antibody. Recently, the digestion and unambiguous sequence coverage of adalimumab was obtained using an about 1 s digestion at 0.2 µg/µL (~1.3 pmol/µL).¹³ A tiered decision tree instrument method on the mass spectrometer was implemented to fragment eluting peptides based on their mass-to-charge ratios and charge state. The decision tree dictated if a precursor was fragmented either by ETD or collisional fragmentation, and whether it was analyzed in the Orbitrap or the ion trap.¹³ Extending this methodology to bsAbs was driven by the increased research on bispecific antibodies in drug development.⁴ However, since each chain of a bsAb is different, they are effectively at half the concentration of the individual chains of a monospecific antibody. Therefore, the digestion of a bsAb can be diffusion limited and result in a greater number of enzymatic cleavages in comparison to a monospecific antibody.

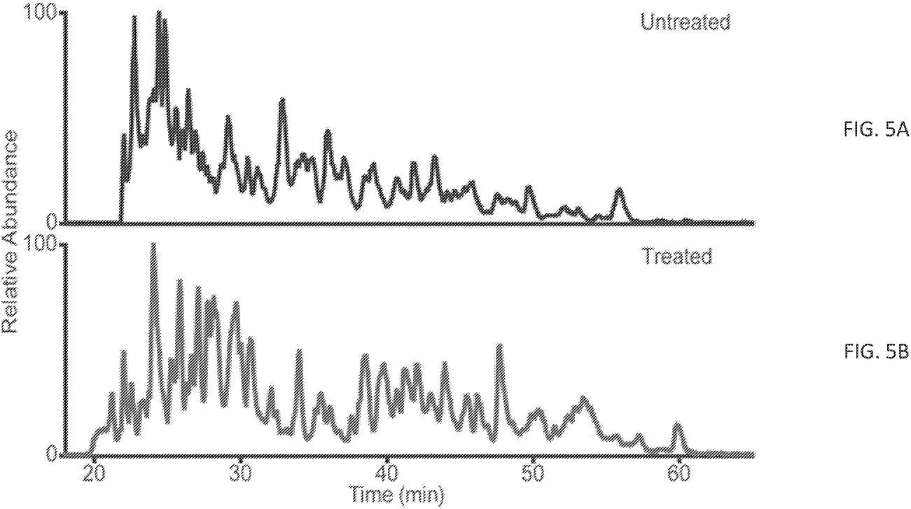

The biosimilar of the bsAb emicizumab was used to demonstrate the diffusion limited digestion of a bsAb.^{19,20} Emicizumab employs a common light chain with a knob-in-hole mutation in the Fc/2 region. Since the concentration of the light chain is twice that of the heavy chains in this bsAb following reduction and alkylation, the effect of concentration should be evident between the subunits. FIG. 5A shows the chromatogram of the bsAb digested at 0.2 µg/µL (about 1.3 pmol/µL). A vast majority of the ion current lies within the first 15 minutes of analysis, which is indicative of a high abundance of small to medium sizes peptides (about 0.8 kDa - about 5 kDa). However, larger peptides can be more desirable for analysis because they can provide more confident connectivity.

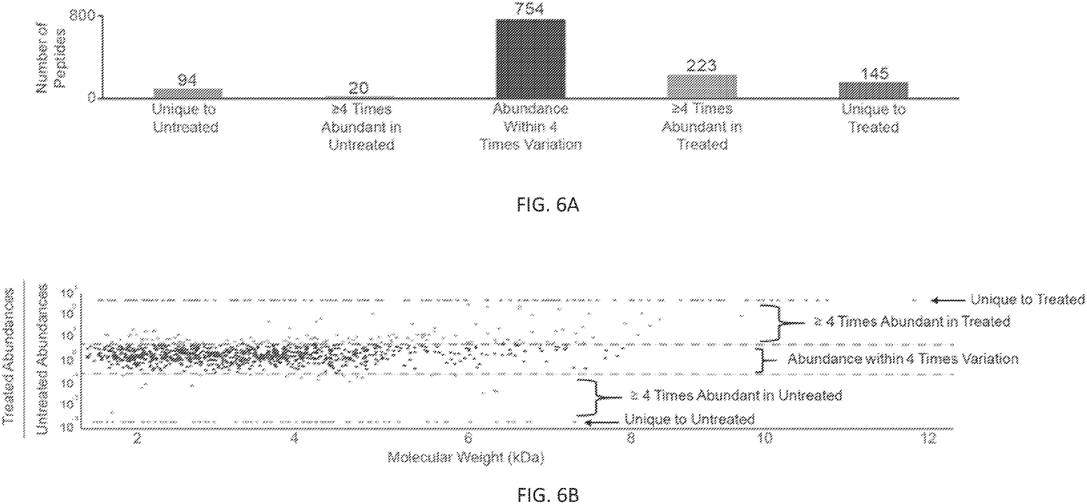

Protamine was introduced to the bsAb sample following reduction and alkylation such that the concentration of both bsAb and protamine was 0.2 µg/µL. This mixture was digested for about 1 s to give the chromatogram shown in FIG. 5B. Qualitatively, the ion current is not as concentrated in the beginning of the gradient (about 20 min - about 28 min) as in the untreated digestion. Additionally, the ion current later in the gradient (about 28 min - about 60 min) increased in relative abundance, indicating that larger peptides were present in a higher abundance than in the untreated sample. All identified peptide abundances were calculated and grouped based on their difference in abundance to define these differences. FIG. 6A shows that a majority of peptides (about 61%) were observed in both digestions at similar abundances (within a 4-fold change). Minor differences in the experimental setup can result in slight changes in the abundances of peptides, such as changes in chromatography, flow rate, and differing ion suppression. These minor differences in abundance were accounted for by using a large window to classify peptides with similar abundances. Significantly, over 200 peptides were observed in the treated digestion that were greater than 4 times more abundant than in the untreated sample. Conversely, only 20 peptides were more abundant in the untreated digestion. While these peptides were still identified in the untreated sample, increasing the abundance of peptides is desirable because it can result in a higher quality fragmentation spectrum. Substantially, 145 unique peptides were observed in the treated digestion and 62 of those peptides were above 5 kDa. Comparatively, only 15 out of the 94 unique peptides in the untreated digestion were above 5 kDa.

FIG. 6B plots the log of the ratio of abundance between the treated and untreated digestion against the molecular weight. The points between the dashed lines are considered similar because they were within a 4-fold change in abundance. Peptides across the molecular weight range increased in abundance after treatment with protamine and are depicted in FIG. 6B as the points outside the dashed lines. Importantly, peptides > 5 kDa significantly increased in abundance. Therefore, treating the sample with protamine provided for larger peptides to be retained at higher abundances due to the reduction in the number of enzymatic cleavages. These larger peptides provide less sequence ambiguity than the smaller peptides produced from the untreated sample.

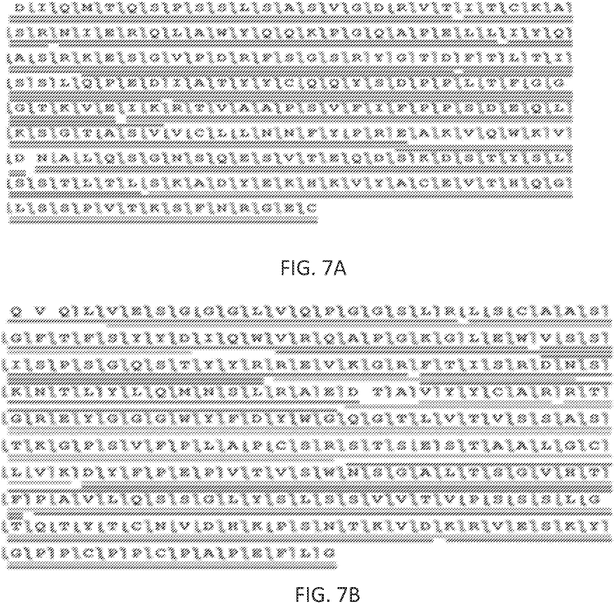

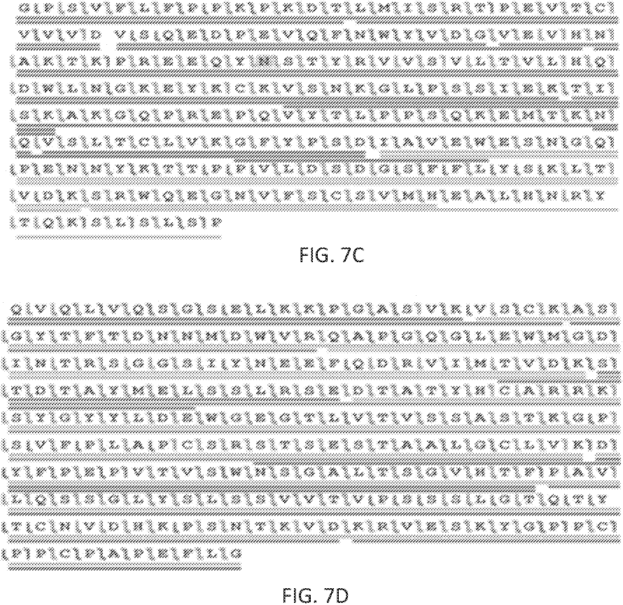

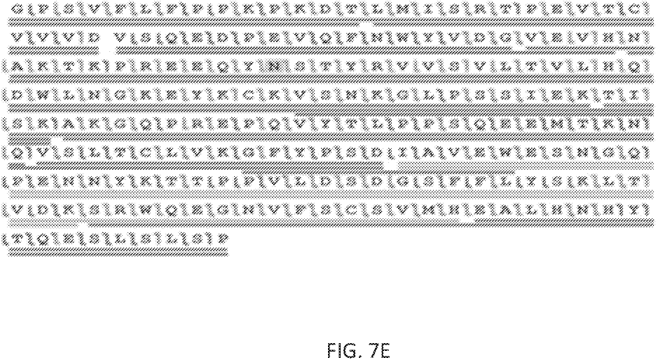

FIGS. 7A-7E show the peptide maps as well as observed fragment ions by ETD (flat hash marks) and collisional fragmentation (angled hash marks; both CAD and HCD). 1,096 of 1,101 fragment ions were observed resulting in about 99.5% sequence coverage. Although protamine treatment successfully retained larger peptides, it did not alter the preferential cleavage of particular residues. For example, shown in FIGS. 7C and 7E, there is a missed fragment cleavage at D29-V30. No peptides were observed that contain these residues within a single peptide. Without being bound to any one theory, this was likely a result of Aspergillopepsin I preferentially cleaving here prior to other sites along the molecule, as previously reported.¹³ These minor preferential cleavages account for two of the five missed cleavages. When comparing this data to previous analyses with the enzyme reactor, a different set of minor preferred cleavages sites was observed due to the high sequence fluctuation in the variable regions of the light and heavy chains.^{12,13}

Summary: The analyte concentration changes the molecular weight distribution of peptides produced from a digestion with the Aspergillopepsin I enzyme reactor. Introducing protamine to the sample mitigated the effect of concentration on the resulting digestion. Since protamines are highly basic proteins containing a large number of Aspergillopepsin I favorable cleavage sites, they were able to block a portion of enzymatic active sites and shift the system closer to a catalytically-limited state. These concentration effects can have biologically relevant consequences considering the concentration of each chain of an IgG-like bispecific antibody is half that of a monospecific antibody. Protamine was used to mitigate these concentration effects so that an IgG-like common light chain bispecific antibody was successfully analyzed. Unambiguous sequence coverage of the antibody was obtained from an about 1 s digestion and a single chromatographic analysis. While this work used primarily about 1 s digestions to demonstrate these concentration effects, controlling the flow rate through the reactor can easily be used to further tune the peptide size distribution. For example, very large proteins (e.g., >30 kDa) can be digested with protamine at very short digestion times (e.g., about 400 ms - about 600 ms) to provide high abundance peptides that contain a significant percentage of the molecule. These large peptides can then be targeted for extensive characterization with techniques such as parallel-ion parking ETD with sequential ion-ion proton transfer (IIPT) since their molecular weights are better suited for the current limitations of available instrumentation. ^{38,39}

### Example 3

### Disulfide Bond Localization of BsAb

Additional Methods: About 4 µg of EpCAM BiTE, a gift from the Cobbold Laboratory at Harvard University (Cambridge, Massachusetts, United States of America) (see also PCT International Publication No. WO 2020/010104, herein incorporated by reference in its entirety) was diluted to a final volume of 80.4 µL of digestion buffer to give a final concentration of ~0.05 µg/µL (~0.91 pmol/µL). This sample was not treated with protamine.

### Bispecific Antibody Digestion With Immobilized Enzyme Reactor

Dried Aspergillopepsin I conjugated beads were used as described above in Example 1. The column bed length was packed to ~3 mm for digestion of the BiTE.

### Data Analysis

An initial search of the disulfide bound BiTE was performed in Proteome Discoverer as described above against a database containing the single protein sequence and the common modifications of oxidation at M and pyro-Glu at N-terminal E. This search was used to identify all peptides that did not contain a cysteine. The BYONIC™ node in Proteome Discoverer does not have the option to search for disulfide bonds. Therefore, the .raw file was converted using in-house software into 4 MGF files consisting of 1) Orbitrap ETD scans, 2) Orbitrap HCD scans, 3) ion trap ETD scans, and 4) ion trap CAD scans. Each MGF file was searched individually using the stand-alone BYONIC™ program (Protein Metrics, Cupertino, California, United States of America) with the disulfide option selected and a cleavage specificity of C-terminal to K,L,R,D,P,E and N-terminal to V,L,S,A,T,I. While Aspergillopepsin I is nonspecific, the search software fails if more amino acids are added to the specificity due to the exponential increase in potential peptide combinations to search. However, since the number of missed cleavages is set to unlimited, the search will still perform a nonspecific search. The ion trap data file searches contained a maximum precursor mass of 6,000 Da while the Orbitrap data file searches contained a maximum precursor mass of 10,000 Da. Selected fragmentation scans were chosen for manual annotation to reconstruct the sequence and characterize the disulfide bonds.

### Discussion

There are a group of bsAbs that eliminate the Fc region of the molecule altogether. One of these types of bsAbs is known as Bispecific T-cell engagers, or BiTEs (42). This molecule removes all chain pairing problems by expressing the molecule as a single peptide chain. Strings of glycines and serines are used to connect the C-terminus of the V_{L} regions to the N-terminus V_{H} regions and the C-terminus of the V_{H} region to the N-terminus of the V_{L} region. This results in a protein that is about 50 kDa -55 kDa in molecular weight.

Functionally, BiTEs are used to bring a T-cell and a cancerous cell in close proximity to trigger an immune response.⁴⁴ Typically, a T-cell receptor recognizes a peptide presented by an MHC molecule as non-self, triggering the immune system to respond. However, cancerous cells can hinder the presentation of the MHC molecules.⁴⁵ The BiTE can mimic the MHC molecule and trigger an immune response by targeting T-cell receptors, the most common being CD3, and a protein in high abundance on cancerous cells. Blinatumomab was the first BiTE approved for use in 2014 under the accelerated approval program for acute lymphoblastic leukemia by targeting CD3 on T-cells and CD19, a receptor on B cells responsible for triggering responses to antigens. ^{46,47}

Although BiTEs are engineered to produce the correct therapeutic molecule, they have a very short half-life. The half-life is roughly 1.25 hours, or about 0.25% of the half-life of a mAb, due to the lack of the Fc region.⁴⁴ Since there is no Fc region, FcRn cannot recognize and prevent the antibody from being eliminated in the bloodstream. The short half-life requires a continuous IV of the BiTE over the 4-8 weeks of treatment. However, the concentration of BiTE required to produce the desired response is fairly low.⁴⁸ This eases the production strain of the BiTE since high concentrations are not needed.

The present example describes the analysis and disulfide bond localization of a BiTE. This particular BiTE targets CD3 and epithelial cell adhesion molecule (EpCAM). EpCAM is primarily located in intercellular spaces of normal cells where tight junctions are formed by epithelial cells. However, EpCAM is homogenous on the surface of cancerous cells. This difference in locality prevents the BiTE from targeting normal cells since they are mainly covered by other cells.⁴⁹

While generating large peptide products is beneficial for maintaining connectivity, there are other instances where smaller peptides are better suited for analysis. One such case is the analysis of disulfide bound peptides. Previously, 17 disulfide bonds in a murine IgG antibody were localized using ta first generation of Aspergillopepsin I enzyme reactor. ¹² However, it took four different digestion times (12 s, 93 s, 260 s, and 740 s) to successfully generate the peptides needed to confidently localize these bonds. The ability for Aspergillopepsin I to cleave was hindered by the antibody higher order structure. The terminal regions were the most exposed, causing these regions to be cleaved first. The accessibility of the antibody shifted towards the center of the molecule as the enzyme cleaved from the termini. The hinge region required the longest digestion time to obtain a peptide of reasonable size for successful analysis.

The previous work with the enzyme reactor for disulfide analysis was carried out at a protein concentration of 0.2 µg/µL.¹² Additionally, selected disulfide bound peptides were targeted for fragmentation after comparison to the reduced analysis of the same digest. As described herein, reducing the concentration of a protein for analysis can increase the number of observed cleavages by Aspergillopepsin I. Therefore, the digestion of a disulfide bound protein into manageable sized peptides can be carried out in a single digestion at a lower concentration. Additionally, leveraging the instrument control software of the Thermo Orbitrap Fusion can provide the ability to fragment the majority of the digestion products in a data-dependent manner, allowing for the analysis of a disulfide bound protein in a single analysis.

Accordingly, a BiTE was used to determine whether reducing the protein concentration and implementing the tiered decision tree method was adequate for analyzing disulfide bound peptides for sequence reconstruction with disulfide bound localization. While this molecule is different than the murine IgG analyzed previously, ¹² it provides a simpler set of parameters to determine if the methodology is sufficient for this type of analysis. Ideally, a successful digestion will generate peptides that are <10 kDa in molecular weight to ensure that only a single cysteine residue is present on a given peptide to better localize the disulfide bond.

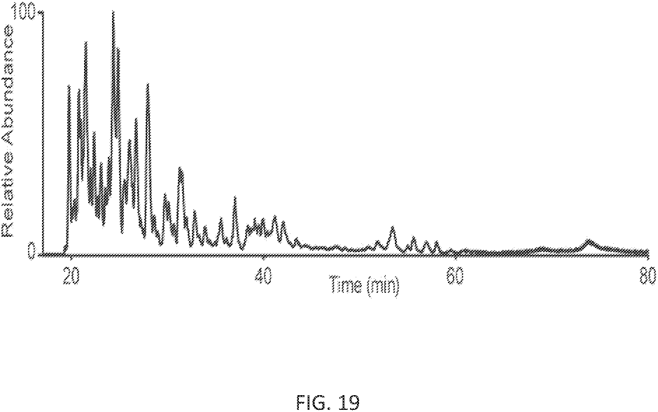

The BiTE was first diluted to 0.05 µg/µL in digestion buffer and then successfully digested by the enzyme reactor in about 1.9 s. FIG. 19 shows the chromatogram of the digestion products. Qualitatively, the majority of the ion current, and thus digestion products, are within the first about 12 minutes of the analysis signifying the generation of small-medium peptides. However, there is still significant ion current for the remainder of the chromatogram. The chromatogram reflects the generation of more manageable sized peptides for disulfide analysis.

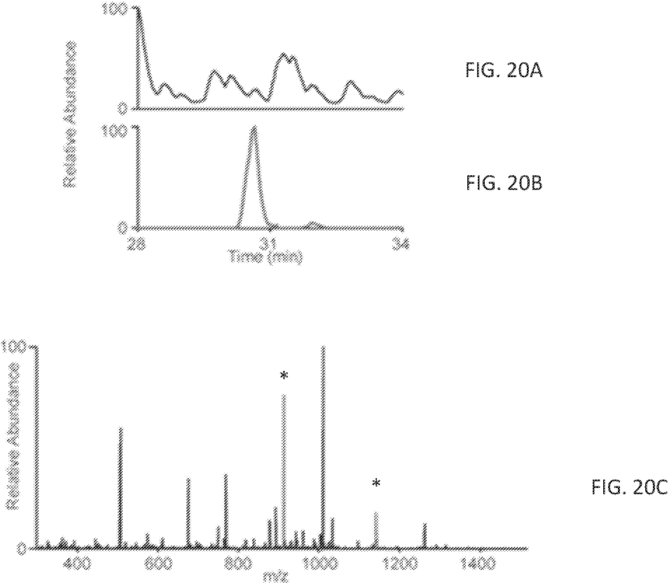

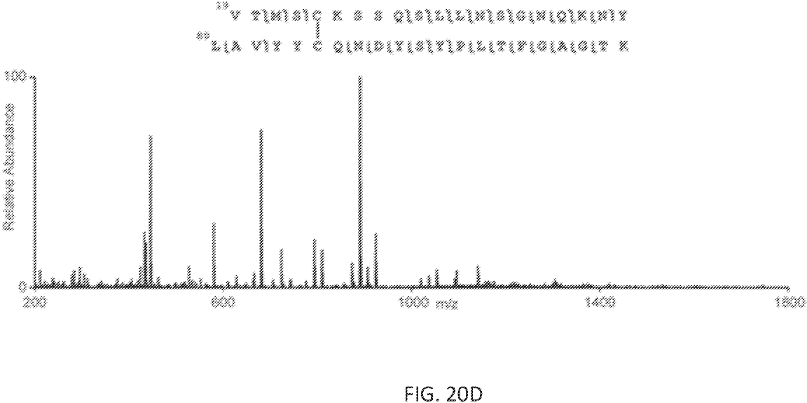

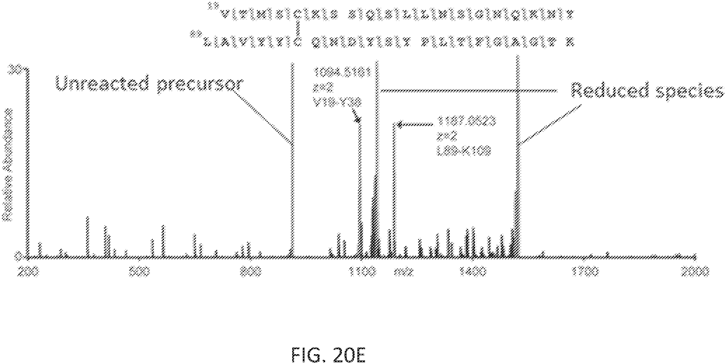

Shown in FIGS. 20A-20E is an example of disulfide bound peptides. These peptides was dissociated by both HCD and ETD using the tiered decision tree data-dependent method on the Thermo Orbitrap Fusion. When dissociating disulfide bound peptides, two ion series will be present in the resulting MS² spectrum. Respectable coverage is generated by the HCD scan with the exception of the residues around the disulfide bound cysteine. See FIG. 20D. Corresponding fragment ions were not observed, preventing the primary structure from being confirmed. However, the disulfide bond can be implied due to the clear presence of two ion series without interference in the MS¹. See FIG. 20C. The ETD scan is shown in FIG. 20E. ETD has an added benefit over HCD in that the disulfide bond can be broken, resulting in two peaks corresponding to the molecular weight of the two bound peptides. ETD obtains coverage throughout each peptide and localizes one of the two cysteine residues.

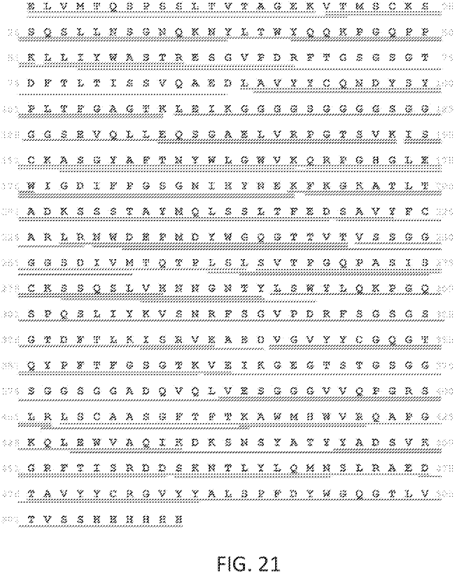

Aspergillopepsin I generated a plethora of peptides to confidently sequence the primary structure as well as characterize the disulfide bonds. FIG. 21 shows 34 peptides that were manually annotated for analysis of these key features. Enzymatic access to some potential cleavage points can be favored because the BiTE maintained tertiary structure during digestion. One such example is at D₃₄₀/V₃₄₁. No peptide contains both of these residues, implying that this is one of the preferred sites of initial cleavage by Aspergillopepsin I.

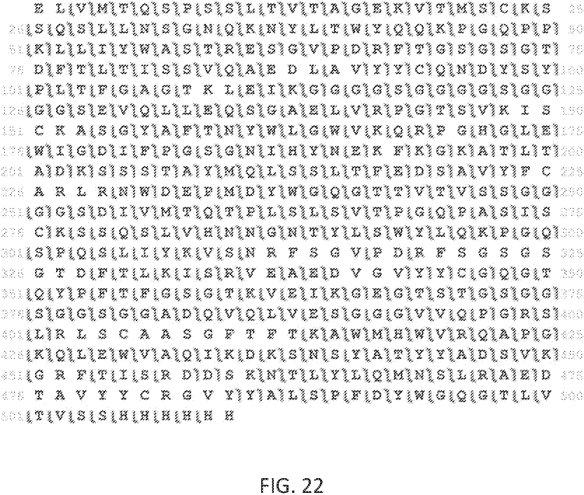

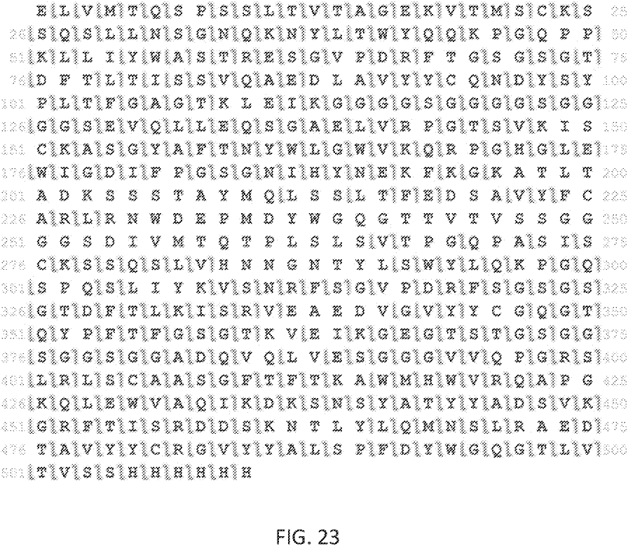

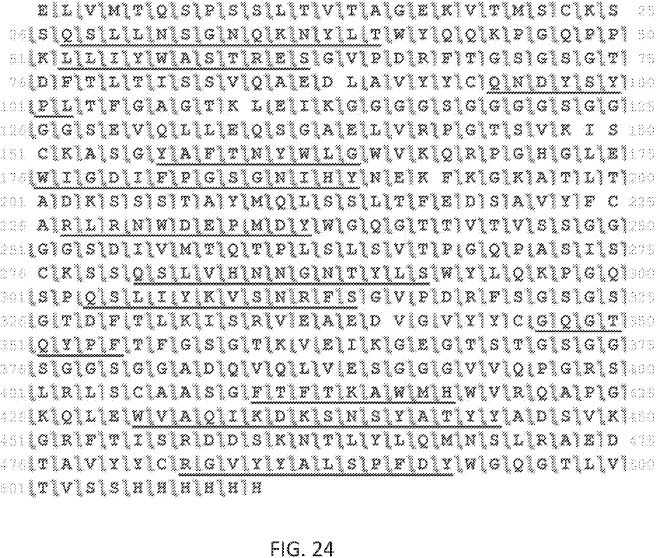

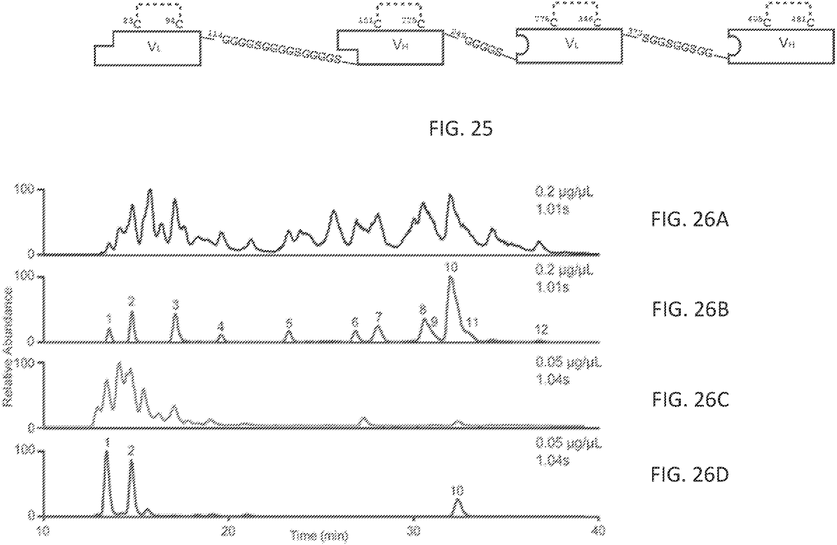

FIGS. 22 and 23 show the collisional fragmentation (both HCD and CAD) and ETD residue cleavages observed, respectively. The collisional fragmentation resulted in 88% residue cleavages while ETD only obtained 76% residue cleavages. This BiTE in particular has stretches of amino acids that lack a basic residue, preventing the ability to obtain high charge states preferable to ETD. The lack of the charge-adding alkylation agent NAEM further inhibits the ability to increase the charge density. However, when these two fragmentation maps are combined (FIG. 24), 98% of the cleavages are observed. Only ten out of a possible 509 cleavages are not observed, resulting in the near complete sequence coverage of this molecule. Additionally, all cysteines were accounted for in the disulfide map. See FIG. 25. The four disulfide bonds, unique to each variable region of the BiTE, were observed with no disulfide scrambling.

The results presented here show that disulfide localization can be performed in a single analysis while maintaining high sequence coverage of the molecule. However, there is a drawback to this experiment: the time it takes to search the data. Since Aspergillopepsin I can cleave between any two amino acids, thousands of potential peptides can be generated. The average search time for a fully reduced, nonspecific search is about 1-2 hours. However, adding disulfide bonds drastically changes the necessary search time. Each termini of disulfide bound peptides can be cleaved anywhere, resulting in the exponential increase in possibilities for the search algorithm to consider and requiring an increase in the search time needed.

Controlling the maximum precursor mass to consider limits the amount of time taken to search the data. Table 3 gives the search time for three data points when searching this data set. A minimal increase in maximum precursor mass results in a substantial increase in search time. Since the search is limited by the molecular weight maximum value, high molecular weight species are unaccounted for in the search. There are peptides observed that are >10 kDa in molecular weight in the analyzed digestion. However, these peptides are unaccounted for in the search algorithm and thus are left unknown without further manual analysis.

TABLE 3

Table of Search Times Required for a Nonspecific Disulfide Analysis.

| Maximum Precursor Mass (kDa) | Search Time (Hours) |
|---|---|
| 8 | 6.7 |
| 9 | 22.0 |
| 10 | 60.5 |

The same issue arises when adding additional PTMs to the search. Only methionine oxidation and pyro-glutamic acid at the protein’s N-terminus were considered for the search. Increasing the number of possible combinations of PTMs will also increase the search time needed. Therefore, if a potentially highly modified nonreduced protein is digested and analyzed, the resulting search should be tailored such that the PTMs can be identified on smaller peptides.

Both the maximum precursor mass and the number of potential PTMs to search greatly inhibit the ability to obtain the full picture of a nonreduced digestion. Searching the data clearly is rate limiting step. However, the ability to obtain near complete sequence coverage and localize the disulfide bonds without scrambling was achieved for the analysis of a novel bsAb.

### Summary

Some experiments require smaller peptides for analysis, such as localizing disulfide bonds. Reducing the concentration of a BiTE induces additional cleavages by Aspergillopepsin I. This results in the successful creation of manageably sized disulfide bound peptides while still maintaining high sequence coverage of the entire molecule.

### Example 4

### Additional Additives

Methods: Digestions of apomyoglobin at 0.05 µg/µL were used to determine if an additive would slow the rate of digestion of apomyoglobin. The additives tested were ubiquitin, β-lactoglobulin (after reduction and alkylation), BSA (after reduction and alkylation), a mixture of the dipeptides RF and KG, penta-lysine (KKKKK), and a mixture protamines from salmon (salmine). 0.2 µg/µL of apomyoglobin was diluted with digestion buffer and an additive to give apomyoglobin a final concentration of 0.05 µg/µL and the additive a concentration of 0.15 µg/µL for ubiquitin, β-lactoglobulin, and BSA and 0.2 µg/µL for all other additives.

Discussion: To reduce the rate of enzymatic digestion of apomyoglobin, further studies were performed by treating a 0.05 µg/µL apomyoglobin solution with an additive protein at three times the mass concentration (0.15 µg/µL). Since there are a greater number of enzymatic sites available for Aspergillopepsin I present within the additive protein, the number of cleavages to apomyoglobin should decrease. Three types of additive proteins were used: 1) a protein lower in molecular weight than apomyoglobin (bovine ubiquitin at ~8.5 kDa), 2) a protein similar in molecular weight to apomyoglobin (bovine β-lactoglobulin at ~18.4 kDa), and 3) a protein larger in molecular weight (bovine serum albumin [BSA] at ~66.4 kDa). Twelve generated apomyoglobin peptides at varying molecular weights were tracked (see FIGS. 26A-26D) to determine if the additive slowed the enzymatic digestion (see Table 4 for list and FIGS. 26B and 26D for chromatographic positions in a 0.2 µg/µL and 0.05 µg/µL apomyoglobin digestion). The larger peptides are eliminated in a diffusional limited system. Therefore, if all the peptides were observed in a treated digestion, the additive was successful at inhibiting the rate of digestion of apomyoglobin.

TABLE 4

Table of Tracked Peptides. The peptide number corresponds to labeled peaks in FIG. 26B. The superscript number prior to the sequence corresponds to peptide’s position in the protein

| Pep . # | M+H (Da) | Sequence | SEQ ID NO: |
|---|---|---|---|
| 1 | 1292.7 0 | ⁴³ FDKFKHLKTE | 13 |
| 2 | 1271.6 6 | ³² LFTGHPETLEK | 14 |
| 3 | 4382.3 2 | ³² LFTGHPETLEKFDKFKHLKTEAEMKASEDLKKHGTWL | 15 |
| 4 | 3242.6 6 | ¹⁰⁷ ISDAIIHVLHSKHPGDFGADAQGAMTKALEL | 16 |
| 5 | 3774.9 0 | ¹⁰⁷ ISDAIIHVLHSKHPGDFGADAQGAMTKALELFRND | 17 |
| 6 | 4051.0 1 | ¹⁰⁵ EFISDAIIHVLHSKHPGDFGADAQGAMTKALELFRND | 18 |
| 7 | 5080.6 1 | ¹⁰⁷ ISDAIIHVLHSKHPGDFGADAQGAMTKALELFRNDIAAKYKELGFQG | 19 |
| 8 | 5356.7 2 | ¹⁰⁵ EFISDAIIHVLHSKHPGDFGADAQGAMTKALELFRNDIAAKYKELGFQG | 20 |
| 9 | 9193.9 5 | ⁷⁰ TALGGILLKKKGHHEAELKPLAQSHATKHKIPIKYLEFISDAIIHVLHSKH- | 21 |
| | | PGDFAGADAQGAMTKALELFRNDIAAKYKELGFQG | |
| 10 | 3403.7 4 | ¹ GLSDGEWQQVLNVWGKVEADIAGHGQEVLIR | 22 |
| 11 | 7767.0 4 | ¹ GLSDGEWQQVLNVWGKVEADIAGHGQEVLIRLFTGHPETLEKFDKF-KHLKTEAEMKASEDLKKHGTVVL | 23 |
| 12 | 4656.3 8 | ¹ GLSDGEWQQVLNVWGKVEADIAGHGQEVLIRLFTGHPETLEK | 24 |

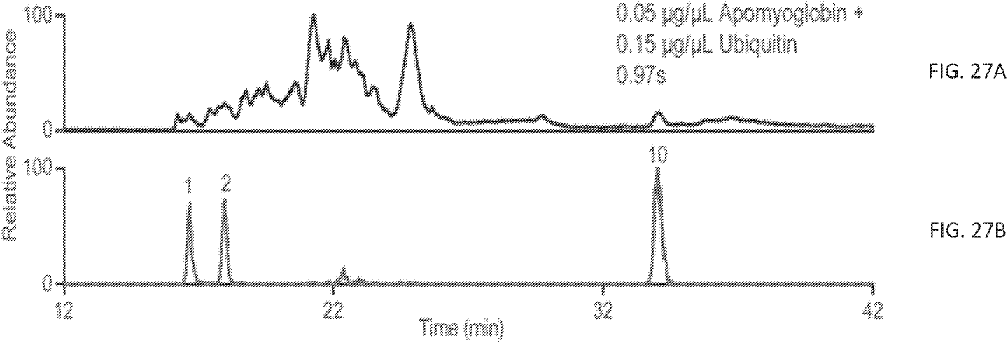

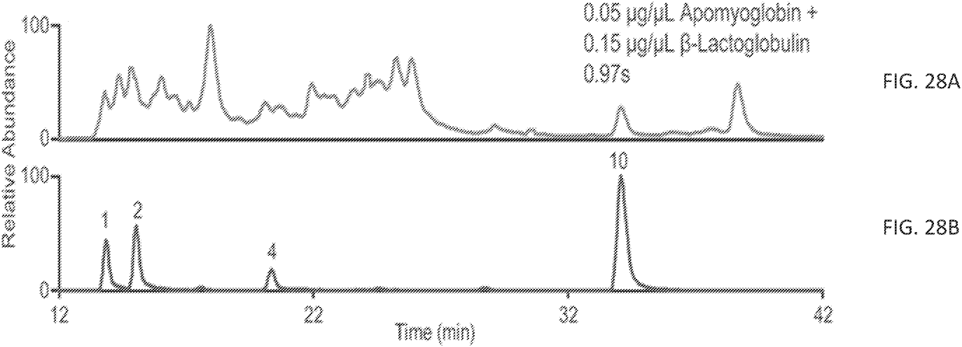

FIGS. 27A-27B and FIGS. 28A-28B show the results of an about 1 s digestion of 0.15 µg/µL ubiquitin + 0.05 µg/µL apomyoglobin (see FIGS. 27A-27B) and of 0.15 µg/µL β-lactoglobulin + 0.05 µg/µL apomyoglobin. See FIGS. 28A-28B. These proteins did not effectively reduce the rate of digestion for apomyoglobin. However, these experiments did reveal an important fact: proteins of similar molecular weight digest regardless of the total protein concentration. Therefore, the individual protein concentration dictates if Aspergillopepsin I will digest that specific protein at a diffusional or catalytic limited rate. This observation becomes relevant when digesting bispecific antibodies, as described in Examples 2 and 3.

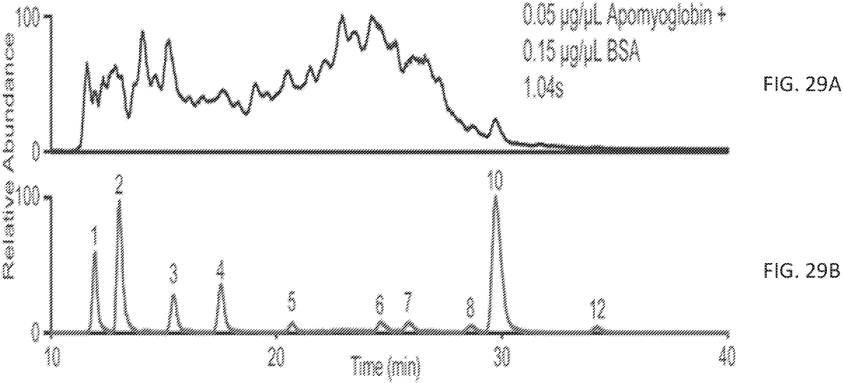

FIGS. 29A-29B show the results of an about 1 s digestion of 0.15 µg/µL BSA + 0.05 µg/µL apomyoglobin. BSA was successful in slowing the rate of digestion of apomyoglobin as seen by the production of the large molecular weight species. Without being bound to any one theory, this was most likely due to the fact that BSA is roughly 4 times larger in molecular weight than apomyoglobin, which in turn affects the relative diffusion rate in the system. Generally, the rate of diffusion is inversely proportional to the molecular weight of the species (i.e. large proteins diffuse slowly).³⁵⁻³⁷ The large molecular weight prevents BSA and its respective digestion products to diffuse quickly enough from the surface of the beads to the bulk solution, thus blocking apomyoglobin from reaching the active sites of Aspergillopepsin I. While BSA was still not the ideal additive, it proved that an additive can be introduced to the digestion mixture to mitigate the diffusional effects.

When protein additives were used, the resulting chromatograms were dominated by digestion products of the more abundant protein. This inhibited the dynamic range and thus the ability to detect low concentration species. Three low molecular weight additives at 0.2 µg/µL were tested: a mixture of the dipeptides RF (about 620.8 pmol/µL) and KG ( about 979.7 pmol/µL), the pentapeptide KKKKK (about 303.26 pmol/µL), and a mixture of protamines from salmon (about 47.4 pmol/µL). These peptides all have a favored site in the P1 position based on the relative enzyme specificity. Additionally, the dipeptides and KKKKK are small and hydrophilic which prevent their retention on a reverse phase column, thereby eliminating the potential for interference with the digestion products of interest. However, in the present study, RF/KG and KKKKK were unsuccessful in reducing the rate of digestion of apomyoglobin. In both cases, only three of the possible 12 peptides (1, 2, and 10) were observed. Since these peptides and any potential digestion products did not retain on the reverse phase column, it cannot be determined using the methods at hand if these peptides interacted with Aspergillopepsin I in any way. As described hereinabove in Example 2, protamines are successful in reducing the rate of digestion of apomyoglobin.

### REFERENCES

All references listed in the instant disclosure, including but not limited to all patents, patent applications and publications thereof, scientific journal articles, and database entries (including but not limited to UniProt, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.

Ecker, D. M.; Jones, S. D.; Levine, H. L. The Therapeutic Monoclonal Antibody Market. MAbs2015, 7 (1), 9-14.

Krishnamurthy, A.; Jimeno, A. Bispecific Antibodies for Cancer Therapy: A Review. Pharmacol. Ther. 2018, 185, 122-134.

Thakur, A.; Huang, M.; Lum, L. G. Bispecific Antibody Based Therapeutics: Strengths and Challenges. Blood Rev. 2018, 32 (4), 339-347.

Labrijn, A. F.; Janmaat, M. L.; Reichert, J. M.; Parren, P. W. H. I. Bispecific Antibodies: A Mechanistic Review of the Pipeline. Nat. Rev. Drug Discov. 2019, 18, 585-608.

Suurs, F. V.; Lub-de Hooge, M. N.; de Vries, E. G. E.; de Groot, D. J. A. A Review of Bispecific Antibodies and Antibody Constructs in Oncology and Clinical Challenges. Pharmacol. Ther. 2019, 201, 103-119.

Brinkmann, U.; Kontermann, R. E. The Making of Bispecific Antibodies. MAbs2017, 9 (2), 182-212.

Zhang, Y.; Fonslow, B. R.; Shan, B.; Baek, M.-C.; Yates III, J. R. Protein Analysis by Shotgun/Bottom-up Proteomics. Chem. Rev. 2013, 113 (4), 2343-2394.

Li, H.; Ortiz, R.; Tran, L.; Hall, M.; Spahr, C.; Walker, K.; Laudemann, J.; Miller, S.; Salimi-Moosavi, H.; Lee, J. W. General LC-MS/MS Method Approach to Quantify Therapeutic Monoclonal Antibodies Using a Common Whole Antibody Internal Standard with Application to Preclinical Studies. Anal. Chem. 2012, 84 (3), 1267-1273.

Bongers, J.; Cummings, J. J.; Ebert, M. B.; Federici, M. M.; Gledhill, L.; Gulati, D.; Hilliard, G. M.; Jones, B. H.; Lee, K. R.; Mozdzanowski, J.; et al. Validation of a Peptide Mapping Method for a Therapeutic Monoclonal Antibody: What Could We Possibly Learn about a Method We Have Run 100 Times? J. Pharm. Biomed. Anal.2000, 21 (6), 1099-1128.

Tsiatsiani, L.; Heck, A. J. R. Proteomics beyond Trypsin. FEBS J. 2015, 282 (14), 2612-2626.

Swaney, D. L.; Wenger, C. D.; Coon, J. J. Value of Using Multiple Proteases for Large-Scale Mass Spectrometry-Based Proteomics. J. Proteome Res. 2010, 9 (3), 1323-1329.

Zhang, L.; English, A. M.; Bai, D. L.; Ugrin, S. A.; Shabanowitz, J.; Ross, M. M.; Hunt, D. F.; Wang, W.-H. Analysis of Monoclonal Antibody Sequence and Post-Translational Modifications by Time-Controlled Proteolysis and Tandem Mass Spectrometry. Mol. Cell. Proteomics2016, 15 (4), 1479-1488.

Hinkle, J.; D’Ippolito, R. A.; Panepinto, M. C.; Wang, W.; Bai, D. L.; Shabanowitz, J.; Hunt, D. F. Unambiguous Sequence Characterization of a Monoclonal Antibody in a Single Analysis Using a Nonspecific Immobilized Enzyme Reactor. Anal. Chem. 2019, 91 (21), 13547-13554.

Ichishima, E. Aspergillopepsin I. Handb. Proteolytic Enzym. 2013, 135-141.

Merchant, A. M.; Zhu, Z.; Yuan, J. Q.; Goddard, A.; Adams, C. W.; Presta, L. G.; Carter, P. An Efficient Route to Human Bispecific IgG. Nat. Biotechnol. 1998, 16 (7), 677-681.

Ridgway, J. B. B.; Presta, L. G.; Carter, P. ‘Knobs-into-Holes’ Engineering of Antibody CH3 Domains for Heavy Chain Heterodimerization. Protein Eng. Des. Sel. 1996, 9 (7), 617-621.

Gunasekaran, K.; Pentony, M.; Shen, M.; Garrett, L.; Forte, C.; Woodward, A.; Ng, S. Bin; Born, T.; Retter, M.; Manchulenko, K.; et al. Enhancing Antibody Fc Heterodimer Formation through Electrostatic Steering Effects. J. Biol. Chem. 2010, 285 (25), 19637-19646.

Krah, S.; Sellmann, C.; Rhiel, L.; Schröter, C.; Dickgiesser, S.; Beck, J.; Zielonka, S.; Toleikis, L.; Hock, B.; Kolmar, H.; et al. Engineering Bispecific Antibodies with Defined Chain Pairing. N. Biotechnol. 2017, 39, 167-173.

Knight, T.; Callaghan, M. U. The Role of Emicizumab, a Bispecific Factor IXa- and Factor X-Directed Antibody, for the Prevention of Bleeding Episodes in Patients with Hemophilia A. Ther. Adv. Hematol. 2018, 9 (10), 319-334.

Lenting, P. J.; Denis, C. V; Christophe, O. D. Emicizumab, a Bispecific Antibody Recognizing Coagulation Factors IX and X: How Does It Actually Compare to Factor VIII? Blood2017, 130 (23), 2463.

von Pawel-Rammingen, U.; Johansson, B. P.; Bjorck, L. IdeS, a Novel Streptococcal Cysteine Proteinase with Unique Specificity for Immunoglobulin G. EMBO J. 2002, 21 (7), 1607-1615.

Earley, L.; Anderson, L. C.; Bai, D. L.; Mullen, C.; Syka, J. E. P.; English, A. M.; Dunyach, J.-J.; Stafford, G. C.; Shabanowitz, J.; Hunt, D. F.; et al. Front-End Electron Transfer Dissociation: A New Ionization Source. Anal. Chem. 2013, 85 (17), 8385-8390.

Martin, S. E.; Shabanowitz, J.; Hunt, D. F.; Marto, J. A. Subfemtomole MS and MS/MS Peptide Sequence Analysis Using Nano-HPLC Micro-ESI Fourier Transform Ion Cyclotron Resonance Mass Spectrometry. Anal. Chem. 2000, 72 (18), 4266-4274.

Udeshi, N. D.; Compton, P. D.; Shabanowitz, J.; Hunt, D. F.; Rose, K. L. Methods for Analyzing Peptides and Proteins on a Chromatographic Timescale by Electron-Transfer Dissociation Mass Spectrometry. Nat. Protoc. 2008, 3, 1709.

Riley, N. M.; Mullen, C.; Weisbrod, C. R.; Sharma, S.; Senko, M. W.; Zabrouskov, V.; Westphall, M. S.; Syka, J. E. P.; Coon, J. J. Enhanced Dissociation of Intact Proteins with High Capacity Electron Transfer Dissociation. J. Am. Soc. Mass Spectrom. 2016, 27 (3), 520-531.

Laidler, K. J.; Bunting, P. S. [9] The Kinetics of Immobilized Enzyme Systems. In Enzyme Kinetics and Mechanism - Part B: Isotopic Probes and Complex Enzyme Systems; Purich, D. L. B. T.-M. in E., Ed.; Academic Press, 1980; Vol. 64, pp 227-248.

Lee, G. K.; Lesch, R. A.; Reilly, P. J. Estimation of Intrinsic Kinetic Constants for Pore Diffusion-limited Immobilized Enzyme Reactions. Biotechnol. Bioeng. 1981, 23 (3), 487-497.

Berendsen, W. R.; Lapin, A.; Reuss, M. Investigations of Reaction Kinetics for Immobilized Enzymes—Identification of Parameters in the Presence of Diffusion Limitation. Biotechnol. Prog. 2006, 22 (5), 1305-1312.

Rodrigues, R. C.; Ortiz, C.; Berenguer-Murcia, A.; Torres, R.; Fernandez-Lafuente, R. Modifying Enzyme Activity and Selectivity by Immobilization. Chem. Soc. Rev. 2013, 42 (15), 6290-6307.

Shuler, M. L.; Kargi, F.; DeLisa, M. Bioprocess Engineering: Basic Concepts; Pearson Education, 2017.

Ausió, J. Histone H1 and Evolution of Sperm Nuclear Basic Proteins. J. Biol. Chem. 1999, 274 (44), 31115-31118.

Lewis, J. D.; Saperas, N.; Song, Y.; Zamora, M. J.; Chiva, M.; Ausió, J. Histone H1 and the Origin of Protamines. Proc. Natl. Acad. Sci. 2004, 101 (12), 4148-4152.

E Kasinsky, H.; Maria Eirin-Lopez, J.; Ausió, J. Protamines: Structural Complexity, Evolution and Chromatin Patterning. Protein Pept. Lett. 2011, 18 (8), 755-771.

Lewis, J. D.; Song, Y.; de Jong, M. E.; Bagha, S. M.; Ausió, J. A Walk Though Vertebrate and Invertebrate Protamines. Chromosoma2003, 111 (8), 473-482.

Young, M. E.; Carroad, P. A.; Bell, R. L. Estimation of Diffusion Coefficients of Proteins. Biotechnol. Bioeng. 1980, 22 (5), 947-955.

Bódalo, A.; Gómez, J. L.; Gómez, E.; Bastida, J.; Iborra, J. L.; Manjón, A. Analysis of Diffusion Effects on Immobilized Enzymes on Porous Supports with Reversible Michaelis-Menten Kinetics. Enzyme Microb. Technol. 1986, 8 (7), 433-438.

He, L.; Niemeyer, B. A Novel Correlation for Protein Diffusion Coefficients Based on Molecular Weight and Radius of Gyration. Biotechnol. Prog. 2003, 19 (2), 544-548.

Compton, P. D.; Zamdborg, L.; Thomas, P. M.; Kelleher, N. L. On the Scalability and Requirements of Whole Protein Mass Spectrometry. Anal. Chem. 2011, 83 (17), 6868-6874.

Schaffer, L. V; Millikin, R. J.; Miller, R. M.; Anderson, L. C.; Fellers, R. T.; Ge, Y.; Kelleher, N. L.; LeDuc, R. D.; Liu, X.; Payne, S. H.; et al. Identification and Quantification of Proteoforms by Mass Spectrometry. Proteomics2019, 19 (10), 1800361.

Evans, S. V.; Brayer, G. D. High-Resolution Study of the Three-Dimensional Structure of Horse Heart Metmyoglobin. J. Mol. Biol. 1990, 213 (4), 885-897. https://doi.org/10.1016/S0022-2836(05)80270-0.

Weiss, M. S.; Palm, G. J.; Hilgenfeld, R. Crystallization, Structure Solution and Refinement of Hen Egg-White Lysozyme at PH 8.0 in the Presence of MPD. Acta Crystallogr. Sect. D2000, 56 (8), 952-958.

Brownlow, S.; Cabral, J. H. M.; Cooper, R.; Flower, D. R.; Yewdall, S. J.; Polikarpov, I.; North, A. C.; Sawyer, L. Bovine β-Lactoglobulin at 1.8 Å Resolution — Still an Enigmatic Lipocalin. Structure1997, 5 (4), 481-495. https://doi.org/10.1016/S0969-2126(97)00205-0.

Fehlhammer, H.; Bode, W.; Huber, R. Crystal Structure of Bovine Trypsinogen at 1 · 8 Å Resolution: II. Crystallographic Refinement, Refined Crystal Structure and Comparison with Bovine Trypsin. J. Mol. Biol. 1977, 111 (4), 415-438.

Huehls AM, Coupet TA, Sentman CL (2015) Bispecific T-cell engagers for cancer immunotherapy. Immunol Cell Biol 93(3):290-296.

Ellerman D (2019) Bispecific T-cell engagers: Towards understanding variables influencing the in vitro potency and tumor selectivity and their modulation to enhance their efficacy and safety. Methods 154:102-117.

Nagorsen D, Baeuerle PA (2011) Immunomodulatory therapy of cancer with T cell-engaging BiTE antibody blinatumomab. Exp Cell Res 317(9):1255-1260.

PrzepiorkaD, et al. (2015) FDA Approval: Blinatumomab. Clin Cancer Res 21(18):4035 LP - 4039.

Bargou R, et al. (2008) Tumor Regression in Cancer Patients by Very Low Doses of a T Cell-Engaging Antibody. Science (80-) 321(5891):974 LP - 977.

Munz M, Baeuerle PA, Gires O (2009) The Emerging Role of EpCAM in Cancer and Stem Cell Signaling. Cancer Res 69(14):5627 LP - 5629.

It will be understood that various details of the presently disclosed subject matter can be changed without departing from the scope of the presently disclosed subject matter. Furthermore, the foregoing description is for the purpose of illustration only, and not for the purpose of limitation.

1. A method for characterizing a protein, said method comprising: disposing said protein in a digestion buffer; disposing a hydrolyzing agent inhibitor in the digestion buffer; passing the digestion buffer comprising said protein and said hydrolyzing agent inhibitor through a reaction chamber comprising at least one hydrolyzing agent, wherein said protein contacts said hydrolyzing agent in the presence of said inhibitor and is present in the chamber for a period of time (t) sufficient to produce protein fragments and provide digestion of said protein in the chamber, wherein the passing of the digestion buffer comprising the protein and the hydrolyzing agent inhibitor through the chamber is done at an adjustable flow rate; and performing multi-segment liquid chromatography tandem mass spectrometry (LC MS/MS) to characterize the protein.

2. The method of claim 1, wherein the protein is denatured to provide a denatured protein before being disposed in the digestion buffer.

3. The method of claim 2, wherein said denatured protein is reduced and alkylated before being disposed in the digestion buffer.

4. (canceled)

5. The method of claim 1, wherein said protein is selected from the group consisting of an antibody, an antibody-like molecule, an antibody light chain, an antibody heavy chain, or biologically active fragments and homologs thereof.

6. (canceled)

7. (canceled)

8. The method of claim 5, wherein said antibody is a bispecific antibody (bsAb).

9. The method of claim 1, wherein disposing the protein in a digestion buffer comprises disposing the protein in the digestion buffer at a first concentration, wherein said first concentration is about 0.2 micrograms per microliter (µg/µL) or less.

10. (canceled)

11. The method of claim 1, wherein the hydrolyzing agent inhibitor comprises a protein, a peptide, or a buffer, optionally wherein the hydrolyzing agent inhibitor comprises at least one inhibitor selected from the group consisting of guanidinium chloride, bovine serum albumin (BSA), and a protamine.

12. The method of claim 11, wherein the hydrolyzing agent inhibitor comprises a protamine.

13. (canceled)

14. The method of claim 1, wherein disposing the protein in a digestion buffer comprises disposing the protein in the digestion buffer at a first concentration, wherein disposing the hydrolyzing agent inhibitor in the digestion buffer comprises disposing the hydrolyzing agent inhibitor in the digestion buffer at a second concentration, and wherein the second concentration is about the same as or greater than the first concentration.

15. (canceled)

16. The method of claim 1, wherein the protein is contacted with the hydrolyzing agent under acidic and highly chaotropic conditions.

17-20. (canceled)

21. The method of claim 1, wherein the hydrolyzing agent is a protease.

22. (canceled)

23. The method of claim 21, wherein the protease is aspergillopepsin I (SEQ ID NO: 1) or a biologically active fragment or homolog thereof.

24. The method of claim 1, wherein said hydrolyzing agent is immobilized.

25. The method of claim 1, wherein t ranges from about 0.2 seconds (s) to about 20 s.

26. (canceled)

27. The method of claim 1, wherein the adjustable flow rate about 50 microliters per minute (µL/min) to about 4.0 µL/min.

28. (canceled)

29. The method of claim 1, wherein the digested protein fragments range from about 3 kilodaltons (kDa) in mass to about 10 kDa in mass.

30. The method of claim 1, wherein said characterization of the protein is selected from the group consisting of sequencing, identifying post-translational modifications (PTM), and identifying a site of a disulfide bond.

31-38. (canceled)

39. A method for identifying the site of a disulfide bond in a protein, said method comprising: disposing said protein in a digestion buffer; passing the digestion buffer comprising said protein through a reaction chamber comprising at least one hydrolyzing agent, wherein said protein contacts said hydrolyzing agent and is present in the chamber for a period of time (t) sufficient to produce protein fragments and digestion of said protein occurs in the chamber, wherein the passing of the digestion buffer comprising the protein through the chamber is done at an adjustable flow rate; and performing multi-segment liquid chromatography tandem mass spectrometry (LC MS/MS) to characterize the protein.

40. The method of claim 39, wherein the protein is an antibody, optionally a bispecific antibody.

41. The method of claim 39, wherein t is less than about 3 seconds (s), optionally wherein t is about 1 s to about 3 s, further optionally wherein t is about 1.9 s.

42. The method of claim 39, wherein said protein is disposed in said digestion buffer at a concentration of about 0.02 µg/µL to about 1 µg/µL, optionally about 0.05 µg/µL.

43. (canceled)

44. The method of claim 39, wherein the method is free of the use of ion-ion proton transfer (IIPT).


SEQUENCE LISTING (30 sequences)

SEQ ID NO: 1 - protein, 394 aa, Aspergillus saitoi
MVVFSKTAALVLGLSTAVSAAPAPTRKGFTINQIARPANKTRTVNLPGLYARSLAKFGGTVPQSVKEAASKGSAVTTPQNNDEEYLTPVTVGKSTLHLDFDTGSADLWVFSDELPSSEQTGHDLYTPSSSATKLSGYSWDISYGDGSSASGDVYRDTVTVGGVTTNKQAVEAASKISSEFVQDTANDGLLGLAFSSINTVQPKAQTTFFDTVKSQLDSPLFAVQLKHDAPGVYDFGYIDDSKYTGSITYTDADSSQGYWGFSTDGYSIGDGSSSSSGFSAIADTGTTLILLDDEIVSAYYEQVSGAQESYEAGGYVFSCSTDLPDFTVVIGDYKAVVPGKYINYAPVSTGSSTCYGGIQSNSGLGLSILGDVFLKSQYVVFNSEGPKLGFAAQA

SEQ ID NO: 2 - protein, 10 aa, Equus caballus
FDKFKHLKTE

SEQ ID NO: 3 - protein, 20 aa, Artificial sequence
  Synthetic sequence
VTMSCKSSQSLLNSGNQKNY

SEQ ID NO: 4 - protein, 15 aa, Artificial sequence
  Synthetic sequence
GGGGSGGGGSGGGGS

SEQ ID NO: 5 - protein, 5 aa, Artificial sequence
  Synthetic sequence
GGGGS

SEQ ID NO: 6 - protein, 9 aa, Artificial sequence
  Synthetic sequence
SGGSGGSGG

SEQ ID NO: 7 - protein, 510 aa, Artificial sequence
  Synthetic sequence
ELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKGGGGSGGGGSGGGGSEVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSGGGGSDIVMTQTPLSLSVTPGQPASISCKSSQSLVHNNGNTYLSWYLQKPGQSPQSLIYKVSNRFSGVPDRFSGSGSGTDFTLKISRVEAEDVGVYYCGQGTQYPFTFGSGTKVEIKGEGTSTGSGGSGGSGGADQVQLVESGGGVVQPGRSLRLSCAASGFTFTKAWMHWVRQAPGKQLEWVAQIKDKSNSYATYYADSVKGRFTISRDDSKNTLYLQMNSLRAEDTAVYYCRGVYYALSPFDYWGQGTLVTVSSHHHHHH

SEQ ID NO: 8 - protein, 33 aa, Oncorhynchus sp.
MPRRRRSSSRPVRRRRRPRVSRRRRRRGGRRRR

SEQ ID NO: 9 - protein, 32 aa, Oncorhynchus sp.
PRRRRRSSSRPIRRRRRPRASRRRRRGGRRRR

SEQ ID NO: 10 - protein, 32 aa, Oncorhynchus sp.
PRRRRSSRRPVRRRRRPRVSRRRRRRGGRRRR

SEQ ID NO: 11 - protein, 32 aa, Oncorhynchus sp.
PRRRRSSSRPVRRRRRPRVSRRRRRRGGRRRR

SEQ ID NO: 12 - protein, 30 aa, Oncorhynchus sp.
PRRRRASRRIRRRRRPRVSRRRRRGGRRRR

SEQ ID NO: 13 - protein, 10 aa, Equus caballus
FDKFKHLKTE

SEQ ID NO: 14 - protein, 11 aa, Equus caballus
LFTGHPETLEK

SEQ ID NO: 15 - protein, 38 aa, Equus caballus
LFTGHPETLEKFDKFKHLKTEAEMKASEDLKKHGTVVL

SEQ ID NO: 16 - protein, 31 aa, Equus caballus
ISDAIIHVLHSKHPGDFGADAQGAMTKALEL

SEQ ID NO: 17 - protein, 35 aa, Equus caballus
ISDAIIHVLHSKHPGDFGADAQGAMTKALELFRND

SEQ ID NO: 18 - protein, 37 aa, Equus caballus
EFISDAIIHVLHSKHPGDFGADAQGAMTKALELFRND

SEQ ID NO: 19 - protein, 47 aa, Equus caballus
ISDAIIHVLHSKHPGDFGADAQGAMTKALELFRNDIAAKYKELGFQG

SEQ ID NO: 20 - protein, 49 aa, Equus caballus
EFISDAIIHVLHSKHPGDFGADAQGAMTKALELFRNDIAAKYKELGFQG

SEQ ID NO: 21 - protein, 86 aa, Equus caballus
TALGGILLKKKGHHEAELKPLAQSHATKHKIPIKYLEFISDAIIHVLHSKHPGDFAGADAQGAMTKALELFRNDIAAKYKELGFQG

SEQ ID NO: 22 - protein, 31 aa, Equus caballus
GLSDGEWQQVLNVWGKVEADIAGHGQEVLIR

SEQ ID NO: 23 - protein, 69 aa, Equus caballus
GLSDGEWQQVLNVWGKVEADIAGHGQEVLIRLFTGHPETLEKFDKFKHLKTEAEMKASEDLKKHGTVVL

SEQ ID NO: 24 - protein, 42 aa, Equus caballus
GLSDGEWQQVLNVWGKVEADIAGHGQEVLIRLFTGHPETLEK

SEQ ID NO: 25 - protein, 214 aa, Artificial sequence
  Synthetic sequence
DIQMTQSPSSLSASVGDRVTITCKASRNIERQLAWYQQKPGQAPELLIYQASRKESGVPDRFSGSRYGTDFTLTISSLQPEDIATYYCQQYSDPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 239 aa, Artificial sequence
  Synthetic sequence
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRTGREYGGGWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFLG

SEQ ID NO: 27 - protein, 209 aa, Artificial sequence
  Synthetic sequence
GPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQKEMTKNQVSLTCLVKGFYPSDIAVEWESNGQPENNYKTTPPVLDSDGSFFLYSKLTVDKSRWQEGNVFSCSVMHEALHNRYTQKSLSLSP

SEQ ID NO: 28 - protein, 235 aa, Artificial sequence
  Synthetic sequence
QVQLVQSGSELKKPGASVKVSCKASGYTFTDNNMDWVRQAPGQGLEWMGDINTRSGGSIYNEEFQDRVIMTVDKSTDTAYMELSSLRSEDTATYHCARRKSYGYYLDEWGEGTLVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFLG

SEQ ID NO: 29 - protein, 209 aa, Artificial sequence
  Synthetic sequence
GPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQESLSLSP

SEQ ID NO: 30 - protein, 21 aa, Artificial sequence
  Synthetic sequence
LAVYYCQNDYSYPLTFGAGTK